(19) United States
(12) Patent Application Publication
(10) Pub. No.: US 2005/0059105 A1
(43) Pub. Date: Mar. 17, 2005

(54) Impedimetric biosensor and its use for rapid detection of bacterial pathogens in solution

(75) Inventors: Evangelyn C. Alocilja, East Lansing, MI (US); Stephen Radke, East Lansing, MI (US)

Correspondence Address: Ian C. McLeod;McLeod & Moyne, P.C., 2190 Commons Parkway, Okemos, MI, 48824, US

(73) Assignee: Board of Trustees of Michigan State University, East Lansing, MI (US)

(21) Appl. No.: 10/895,664

(22) Filed: Jul. 21, 2004

Related U.S. Application Data
(60) Provisional application No. 60/490,105, filed on Jul. 25, 2003.

(51) Int. Cl. C12Q 1/68; G01N 33/554; G01N 33/569; C12M 1/34

(52) U.S. Cl. 435/7.32; 435/287.1; 435/287.2; 205/777.5

(57) ABSTRACT

A method and device for the rapid detection of microorganisms by detection of a change of impedance as a result of the microorganism being bound between interdigitated electrodes is described. In particular, the present invention relates to a handheld device to be used for the detection and, in many instances, the identification of the microorganism.

### CROSS-REFERENCE TO RELATED APPLICATION

This applications claims priority to Provisional Application Ser. No. 60/490,105, filed Jul. 25, 2003.

### STATEMENT REGARDING FEDERALLY SPONSORED RESEARCH OR DEVELOPMENT

The present invention was supported in part by United States Department of Agriculture Grant No. 00.38420.8839. The U.S. government has certain rights in the present invention.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to a method and device for the rapid detection of microorganisms by detection of a change of impedance as a result of the microorganism being bound between spaced apart electrodes. In particular, the present invention relates to a device to be used for the detection and, in many instances, the identification of the microorganism.

(2) Description of Related Art

Pathogenic bacteria and other microorganisms are ubiquitous in the environment. Bacterial pathogens are found in soil, animal intestinal tracts and in fecal-contaminated water. Human beings, on average, harbor more than 150 types of bacteria inside and outside of the body (Madigan et al., Brock Biology of Microorganisms, 8th edition. Upper Saddle River, N.J.: Viacom (1997)). Although many microorganisms are harmless, some are known to be the causative agent of many different infectious diseases including botulism, cholera, diarrhea, emesis, pneumonia and typhoid fever. More than 200 known diseases are transmitted through food and drink alone (Mead et al., In Emerging Infectious Diseases, Vol 5. (1999)).

Although recent data suggests naturally occurring cases of food borne disease outbreaks are declining in the U.S. (Centers for Disease Control (CDC), Report on the Decline of Food borne Illness. Atlanta, Ga. (2002)), it is estimated that food borne diseases cause approximately 76 million illnesses, including 325,000 hospitalizations and 5,000 deaths in the US each year (Mead et al., In Emerging Infectious Diseases, Vol 5. (1999)). Of these, known pathogens account for an estimated 14 million illnesses, 60,000 hospitalizations, and 1,800 deaths indicating pathogens are still a substantial source of infectious disease.

Escherichia coli are bacteria that naturally occur in the intestinal tracts of humans and warm-blooded animals to help the body synthesize vitamins. A particularly dangerous type is referred to as enterohemorrhagic E. coli O157:H7 (EHEC). In 2000, EHEC was the etiological agent in 69 confirmed outbreaks (twice the number in 1999) involving 1564 people in 26 states (CDC, Outbreaks caused by Shiga toxin-producing Escherichia coli, Summary of 2000 Surveillance Data. Atlanta, Ga. (2001)). Of known vehicles, 69% were attributed to food sources, 11% to animal contact, 11% to water exposures, and 8% to person-to-person transmission. Past outbreaks have also been traced to contaminated well water and improperly disinfected swimming pools (Keane et al., New Eng. J. Med. 331: 579-584 (1994)).

EHEC produces toxins that damage the lining of the intestine, cause anemia, stomach cramps and bloody diarrhea, and a serious complication called hemolytic uremic syndrome (HUS) and thrombotic thrombocytopenic purpura (TTP). In North America, HUS is the most common cause of acute kidney failure in children, who are particularly susceptible to this complication. TTP has a mortality rate as high as 50% among the elderly. Recent food safety data indicates that cases of EHEC are rising both in the US and in other industrialized nations (Käferstein et al., Food borne Disease Control: A Transnational Challenge. Geneva, Switzerland: World Health Organization (1997)).

Most cases of EHEC have been traced back to individuals having direct contact with food in situations involving food handling or food preparation. In addition to human contamination, E. coli O157:H7 may be introduced into food through meat grinders, knives, cutting blocks and storage containers. Regardless of source, E. coli O157:H7 has been traced to a number of food products including meat and meat products, game meat, apple juice or cider, milk, alfalfa sprouts, unpasteurized fruit juices, lettuce, game meat, and cheese curds (Doyle et al., In Food Microbiology Fundamentals and Frontiers, 171-191. M. Doyle, L Beuchat, T. Montville eds. Washington, D.C.: American Society for Microbiology (1997); Food And Drug Administration (FDA), FDA/CFSAN Bad Bug Book. http://vm.cfsan.fda.gov/˜mow/chap15.html. (2001)).

Biosensors to detect disease-causing agents in food and water are needed to ensure continued safety of the nation's food supply. The detection and identification of food borne pathogens and other contaminants in raw food materials, food products, processing and assembly lines, hospitals, ports of entry and drinking water supplies continue to rely on conventional culturing techniques. Conventional methods involve enriching the sample and performing various media-based metabolic tests (agar plates or slants) These are elaborate and typically require 2-7 days to obtain results.

Enzyme linked immunosorbent assay (ELISA), polymerase chain reaction (PCR) and hybridization, flow cytometry, molecular cantilevers, matrix-assisted laser desorption/ionization, immunomagnetics, artificial membranes, and spectroscopy are some existing rapid detection technologies (Food Manufacturing Coalition, Real Time Monitoring of Food borne Pathogens: State-of-the-Art Report, P.O. Box 741, Great Falls, Va. 22066 (1997)). ELISA methods for determining and quantifying pathogens in food have been well established (Cohn, SPIE Proceedings Vol. 3259. Bellingham, Wash.: The International Society for Optical Engineering (1998)). The PCR method is extremely sensitive but requires pure samples and hours of processing along with expertise in molecular biology (Meng et al., J. Food Microbiol. 32: 103-113 (1996), Sperveslage et al., J. Microbiol. Meth. 26: 219-224 (1996)). Flow cytometry is another highly effective means for rapid analysis of individual cells at rates up to 1000 cells/sec (McClelland and Pinder, Appl. Environ. Microbiol. 60: 4255-4262 (1994)), though it has been used almost exclusively for eukaryotic cells. These test methods, however, are completed in a microbiology laboratory and are not suitable for on-site monitoring.

Detection techniques for laboratory use cannot adequately serve the needs of health inspectors and monitoring agents in the field. The systems are costly, require specialized training, have complicated processing steps in order to culture or extract the pathogen from food samples, and are time consuming. In comparison, a field-ready biosensor is easy to use, portable and provides results in minutes. Biosensors often operate in a reagentless process enabling the creation of user friendly and field ready devices. Biosensors are analytical instruments possessing a capturing molecule as a reactive surface in close proximity to a transducer, which converts the binding of an analyte to the capturing molecule into a measurable signal (D' Souza, Biosensors and Bioelectronics 16: 337-353 (2001)).

Optical, piezoelectric (PZ) and electrochemical sensing architectures are the main analytical techniques used in biosensors for food and water. An integrated optic interferometer for detecting Salmonella Typhimurium has been developed with sensitivity of 10⁵-10⁷ CFU/mL (Seo et al., J. Food Protect. 62: 431-437 (1999)). A fiber optic evanescent wave biosensor was reported to detect Salmonella, Listeria, and Vibrio species as low as 10² CFU/mL in 20 minutes (Hoyle, ASM News 67: 434-435 (2001)). A surface plasmon resonance biosensor was reported to detect E. coli O157:H7 in meat and environmental samples (Fratamico et al., Biotechnol. Tech. 12: 571-576 (1998); Meeusen et al., Detection of E. coli O157:H7 using a surface plasmon resonance biosensor. Proceedings of the ASAE Meeting (paper no. 01-7030), St. Joseph, Mich.: ASAE (2001)). A luminescence-based method is able to detect 10²-10³ cfu of E. coli in fresh produce (Mathew and Alocilja, Photon based sensing of pathogens in food. Proceedings of the IEEE Sensors Conference, Orlando, Fla., 12-14 June 2002. Submitted to the Transactions of the ASAE; in review (2002)). A portable evanescent-wave fiber-optic biosensor was used to detect Escherichia coli O157:H7 in samples of ground beef in 25 minutes and a concentration as low as 5×10² CFU/mL (DeMarco et al., J. Food Protect. 65: 596-602 (2002)). A quartz crystal microbalance sensor coated with a thin culture medium was able to detect Staphylococcus epidermidis in the range of 10² CFU/mL (Bao et al., Anal. Chem. Acta 319: 97-101 (1996)). Another PZ biosensor, based on flow injection, was able to detect Salmonella Typhimurium concentrations as low as 5.3×10⁵ CFU/mL in 25 minutes. The disadvantages of the above referenced sensors are the long incubation time of the bacteria, the inherent limitations of μL-sized samples when testing food and water, the numerous washing and drying steps required and lack of sensitivity caused by the interference of the food matrix.

Electrochemical biosensors, often referred to as amperometric, conductometric or impedimetric, have the advantage of being highly sensitive, rapid and inexpensive (Sergeyeva et al., Sensors and Actuators B 34: 283-288 (1996); Ghindilis et al., Biosensors and Bioelectronics 13: 113-131 (1998); Bashir and Gomez, Biomedical Microdevices 3: 201-209 (2001)). They measure the change in electrical properties of electrode structures as cells become entrapped or immobilized on or near the electrode. An amperometric immunoassay was also developed for S. aureus utilizing antibodies bound to a carbon electrode and enzyme amplifiers to amplify the detection signal (Rishpon and Ivnitski, Biosensors and Bioelectronics 12: 195-204 (1997)). This biosensor was able to detect S. aureus concentrations down to 1000 CFU/mL in a time of 30 minutes. Brooks et al. (J. Appl. Bacteriol. 73: 189-196 (1992)) developed a sensor capable of detecting S. aureus and Salmonella in pure cultures and in foods down to a concentration of 1-5 CFU/mL after non-selective enrichment. Using porous filter membranes, a flow-through conductometric immuno-filtration biosensor was developed for the detection of Escherichia coli O157:H7 in liquid media (Abdel-Hamid et al., Biosensors and Bioelectronics 14: 309-316 (1998); Muhammad-Tahir and Alocilja, A conductimetric immunosensor for biosecurity. Biosensors and Bioelectronics. Proceedings of the Seventh World Congress on Biosensors, Kyoto, Japan, May 15-17, 2002. Submitted to Biosensors and Bioelectronics; accepted for publication, 2002a, and Fabrication of a membrane strip immunosensor. Proceedings of the IEEE Sensors Conference, Orlando, Fla., 12-14 June 2002. Submitted to the IEEE Sensors Journal, accepted for publication, 2002b (2002)). Impedance spectroscopy has been used successfully (Van Gerwen et al., Sensors and Actuators B 49: 73-80 (1998)) to detect the presence of glucose oxidase (GOD) binding to interdigitated electrodes deposited on silicon oxide (SiO₂) surfaces. The use of impedance spectroscopy to measure the properties of supported lipid bilayer membranes (Wiegand et al., J. Phys. Chem. B 106: 4245-4254 (2002)) immobilized on electrodes shows the ability of impedance methods to discriminate the electrical current in different bilayers. Sheppard et al. (Biosensors and Bioelectronics 11: 967-979 (1995)) showed that it is possible to detect urea concentrations as low as 50 μM on interdigitated electrodes with immobilized urease.

U.S. Pat. No. 4,822,566 to Newman, U.S. Pat. No. 5,567,301 to Setter et al., U.S. Pat. No. 5,958,791 to Roberts et al., U.S. Pat. No. 6,447,657 B1 to Bhullar et al., U.S. Pat. No. 6,537,498 B1 to Lewis et al. and published patent applications 2001/0053535 A1 to Setter et al., 2002/0192653 A1 to Bashir et al., and 2003/0036054 A1 to Ladisch et al. also describe biosensors.

The above biosensors are useful for identifying analytes such as microorganisms in very small sample volumes. None of the above appears to disclose a biosensor which would be useful for detecting microorganisms in large bulk volumes. Therefore, a need remains for a device which would enable detection of microorganisms in large bulk volumes.

### OBJECTS

It is therefore an object of the present invention to provide a handheld device for the detection and ultimately identification of microorganisms in large bulk solutions. In particular, it is an object of the present invention to provide a device which is inexpensive to construct and which is reliable in both a laboratory setting and in field settings.

These and other objects of the present invention will become increasingly apparent with reference to the following drawings and preferred embodiments.

### SUMMARY OF THE INVENTION

The present invention relates to a device for detecting a microorganism which comprises (a) a holder means with an electrode means; and (b) a detection sensor mounted on the holder means in contact with the electrical means, the sensor having spaced apart interdigitated electrodes providing a capacitor and with a capture reagent for the microorganism at least between the electrodes, wherein widths of a space between the electrodes and of the electrodes are each about 5 microns or less, and wherein when the microorganism in a liquid is captured by the capture reagent, the detection sensor is interrogated with an electric circuit producing a change of impedance from the detection sensor without the bound microorganism, thereby detecting the presence of the microorganism.

The present invention further relates to a method for detecting a microorganism which comprises (a) providing a device which comprises a holder means with an electrode means; a detection sensor mounted on the holder means in contact with the electrode means, the sensor having spaced apart interdigitated electrodes providing a capacitor and with a capture reagent for the microorganism at least between the electrodes and wherein widths of spaces between each of the electrodes and of the electrodes are each about 5 microns or less, and wherein when the microorganism in a liquid is captured by the capture reagent, the detection sensor is interrogated with an electric circuit producing a change of impedance from the detection sensor without the bound microorganism, thereby detecting the presence of the microorganism; (b) inserting the detection sensor into a liquid suspected of containing the microorganism; (c) washing the biosensor to remove any unbound material; and (d) determining the change of impedance from the detection sensor by interrogating the detection sensor with the electronic circuit to thereby detect the microorganisms.

The present invention also relates to a device for detecting an analyte in a solution, which comprises a biosensor comprising (i) a substrate material having a surface; (ii) an interdigitated array of at least two electrodes disposed on the surface of the substrate wherein a width of a space between the electrodes and of the electrodes are each about 5 microns or less; and (iii) at least one covalently bound antibody which is specific for the analyte immobilized at least on the surface of the substrate in the space between the electrodes.

The present invention also relates to a method for detecting an analyte in a liquid sample, which comprises (a) providing a device comprising a biosensor comprising (i) a substrate material having a surface; (ii) an interdigitated array of at least two electrodes disposed on the surface of the substrate wherein widths of a space between the electrodes and of the electrodes are each about 5 microns or less in width; and (iii) at least one covalently bound antibody which is specific for the analyte immobilized at least on the surface of the substrate in the space between the electrodes; and (b) immersing the biosensor of the device in the liquid sample for a time sufficient for the antibody to bind the analyte; (c) removing the biosensor of the device from the liquid sample and immersing the biosensor of the device in a neutral solution; and (d) applying a current to the biosensor of the device and measuring the impedance of the current wherein an increase in the impedance indicates the liquid sample has the analyte.

The present invention also relates to a method for detecting a microorganism in a liquid sample, which comprises (a) providing a device comprising a biosensor comprising (i) a substrate material having a surface; (ii) an interdigitated array of at least two electrodes disposed on the surface of the substrate wherein widths of a space between the electrodes and of the electrodes are each about 5 microns or less in width; (iii) at least one covalently bound antibody which is specific for the analyte immobilized at least on the surface of the substrate in the space between the electrodes; and (b) immersing the biosensor of the device in the liquid sample for a time sufficient for the antibody to bind the microorganism; (c) removing the biosensor of the device from the liquid sample and immersing the biosensor of the device in a neutral solution; and (d) applying a current to the biosensor of the device and measuring the impedance of the current wherein an increase in the impedance indicates the liquid sample has the microorganism.

The present invention also relates to a method for identifying a microorganism in a liquid sample, which comprises (a) providing a device comprising a biosensor comprising (i) a substrate material having a surface; (ii) an interdigitated array of at least two electrodes disposed on the surface of the substrate wherein widths of a space between the electrodes and of the electrodes are each about 5 microns or less; and (iii) at least one covalently bound antibody which is specific for the analyte immobilized at least on the surface of the substrate in the space between the electrodes; and (b) immersing the biosensor of the device in the liquid sample for a time sufficient for the antibody to bind the microorganism; (c) removing the biosensor of the device from the liquid sample and immersing the biosensor of the device in water; and (d) applying a current to the biosensor of the device and measuring the impedance of the current wherein an increase in the impedance to a particular amount identifies the microorganism.

In a further embodiment of the device, the substrate material is a planar semiconductor device, for example, the substrate material is a silicon wafer with an insulator layer such as silicon dioxide passivated thereon.

In a further embodiment of the device, the interdigitated electrodes comprise a metal selected from the group consisting of platinum, palladium, gold, indium tin oxide, iridium, rhodium, osmium, copper, silver, and mixtures thereof. In particular aspects of the device, a size of the surface between the interdigitated electrodes corresponds to a size range of at least one of the target analyte (microorganism), preferably wherein the size is about 4 μm to 4000 nm.

In various aspects of the device, the antibody is immobilized on the surface via a heterobifunctional crosslinker or a homobifunctional crosslinker, for example, wherein the heterobifunctional crosslinker is an N-γ-maleimidobutyryloxy succinimide ester. In various embodiments of the device, the antibodies are recombinant antibodies such as recombinant antibodies selected from the group consisting of scFV polypeptides and V_{H} chain polypeptides

In a preferred embodiment of the device, the support means is an elongated probe.

The present invention further provides a method for detecting an analyte such as bacteria, parasites, viruses, toxins, or enzymes in a liquid sample, which comprises (a) providing a device comprising a biosensor comprising (i) a substrate material having a surface; (ii) an interdigitated array of at least two electrodes disposed on the surface of the substrate; (iii) at least one covalently bound antibody which is specific for the analyte immobilized at least on the surface of the substrate between the electrodes; and (iv) an elongated support means with a proximal end for gripping and a distal end for attaching the biosensor, wherein the support means includes for each of the electrodes a conductor which connects the electrodes to an electric circuit comprising an impedance detection means; (b) immersing the biosensor of the device in the liquid sample for a time sufficient for the antibody to bind the analyte; (c) removing the biosensor of the device from the liquid sample and immersing the biosensor of the device in a neutral solution; and (d) applying a current to the biosensor of the device and measuring the impedance of the current wherein an increase in the impedance indicates the liquid sample has the analyte.

The present invention further provides a method for detecting a microorganism in a liquid sample, which comprises (a) providing a device comprising a biosensor comprising (i) a substrate material having a surface; (ii) an interdigitated array of at least two electrodes disposed on the surface of the substrate; (iii) at least one covalently bound antibody which is specific for the analyte immobilized at least on the surface of the substrate between the electrodes; and (iv) an elongated support means with a proximal end for gripping and a distal end for attaching the biosensor, wherein the support means includes for each of the electrodes a conductor which connects the electrodes to an electric circuit comprising an impedance detection means; (b) immersing the biosensor of the device in the liquid sample for a time sufficient for the antibody to bind the microorganism; (c) removing the biosensor of the device from the liquid sample and immersing the biosensor of the device in a neutral solution; and (d) applying a current to the biosensor of the device and measuring the impedance of the current wherein an increase in the impedance indicates the liquid sample has the microorganism.

The present invention further provides a method for identifying a microorganism in a liquid sample, which comprises (a) providing a device comprising a biosensor comprising (i) a substrate material having a surface; (ii) an interdigitated array of at least two electrodes disposed on the surface of the substrate; (iii) at least one covalently bound antibody which is specific for the analyte immobilized at least on the surface of the substrate between the electrodes; and (iv) an elongated support means with a proximal end for gripping and a distal end for attaching the biosensor, wherein the support means includes for each of the electrodes a conductor which connects the electrodes to an electric circuit comprising an impedance detection means; (b) immersing the biosensor of the device in the liquid sample for a time sufficient for the antibody to bind the microorganism; (c) removing the biosensor of the device from the liquid sample and immersing the biosensor of the device in water; and (d) applying a current to the biosensor of the device and measuring the impedance of the current wherein an increase in the impedance to a particular amount identifies the microorganism.

In a further embodiment of the method, the substrate material is a planar semiconductor device, for example, the substrate material is a silicon wafer with an insulator layer such as silicon dioxide passivated thereon.

In a further embodiment of any one of the above methods, the interdigitated electrodes comprise a metal selected from the group consisting of platinum, palladium, gold, iridium, rhodium, osmium, copper, silver, and mixtures thereof. In particular aspects of any one of the above methods, a size of the surface between the interdigitated electrodes corresponds to a size range of at least one of the covalently bound antibodies, preferably wherein the size is about 4 μm to 4000 nm.

In various aspects of any one of the above methods, the antibody is immobilized on the surface via a heterobifunctional crosslinker or a homobifunctional crosslinker, for example, wherein the heterobifunctional crosslinker is an N-γ-maleimidobutyryloxy succinimide ester. In various embodiments of any one of the above methods, the antibodies are recombinant antibodies such as recombinant antibodies selected from the group consisting of scFV polypeptides and V_{H} chain polypeptides

In a preferred embodiment of any one of the above methods, the support means is an elongated probe.

In a further embodiment of any one of the above methods the microorganism is a bacterium such as Escherichia coli strain O157:H7 or other pathogenic bacterium such as Salmonella.

### DESCRIPTION OF THE DRAWINGS

FIG. 1 is a schematic view of the handheld device 200 with biosensor 10 showing steps 1 to 4. Step 1, insert biosensor 10 into test sample solution. Step 2, leave biosensor 10 in solution to allow antibody-antigen to bind together (2-3 minutes). Step 3, remove biosensor 10 from test sample. Step 4, insert the biosensor 10 into a sterile solution. Apply electrical field to interdigitated electrodes on the biosensor 10 and measure the impedance (2 minutes). Total Time is about 5 minutes.

FIG. 2 is a schematic enlarged view of a section of the biosensor 10. The bacteria (or other microorganisms) are immobilized by antibodies between the electrodes 14, 13 using antibodies as the capture reagent. The presence of the bacteria influences the electrical field between the electrodes 14, 13, inducing a change in impedance.

FIG. 3A is a schematic view of a biosensor 220 (measuring 8.0×12.0 mm) showing electrodes 221 and and 223 and electrode 222 forming an interdigitated array 225.

FIG. 3B is an exploded view of the interdigitated array 225 shown in FIG. 3A showing electrodes 221, 222, and 223.

FIG. 3C is an enlarged view of a section of FIG. 3B showing the spacing of the electrodes 221 and 222 in the interdigitated array 225.

FIG. 4 is perspective view of an embodiment of the biosensor 10.

FIG. 5 is cross-section view of the electrode array 34 along line 5 in FIG. 4.

FIG. 6 is a plan view of the handheld device 200.

FIG. 7 is a cross-section view of the electrode array 34 of the biosensor 10 showing the various layers comprising the biosensor 10.

FIG. 8 is a schematic view showing the coupling of an antibody between the electrodes 13, 14. The coupling uses GMBS and MDS to form the coupling agent as a link to the substrate which is typically an inorganic oxide such as silica.

FIG. 9A is a scanning electron microscope (SEM) image showing the unmodified gold electrodes and oxide surface of the biosensor.

FIG. 9B is an SEM image showing the gold electrodes and oxide surface of the biosensor modified with crosslinkers and antibodies after silanization.

FIG. 9C is an SEM image showing the gold electrodes and oxide surface of the biosensor modified with crosslinkers and antibodies after silanization after being testing in a buffer solution containing a concentration of 10² CFU/mL of generic E. coli.

FIG. 9D is an SEM image showing the gold electrodes and oxide surface of the biosensor modified with crosslinkers and antibodies after silanization after being testing in a buffer solution containing a concentration of 10⁶ CFU/mL of generic E. coli.

FIG. 9E is an SEM image showing a close-up view of the unmodified gold electrodes and oxide surface of the biosensor.

FIG. 9F is an SEM image showing a close-up view of the gold electrodes and oxide surface of the biosensor modified with crosslinkers and antibodies after silanization.

FIG. 9G is an SEM image showing the gold electrodes and oxide surface of the biosensor modified with crosslinkers and antibodies after silanization after being testing in a buffer solution containing a concentration of 10² CFU/mL of generic E. coli.

FIG. 9H is an SEM image showing the gold electrodes and oxide surface of the biosensor modified with crosslinkers and antibodies after silanization after being testing in a buffer solution containing a concentration of 10⁶ CFU/mL of generic E. coli.

FIG. 9I is an SEM image showing close-up view of the unmodified gold electrodes and oxide surface of the biosensor.

FIG. 9J is an SEM image showing a close-up view of the gold electrodes and oxide surface of the biosensor modified with crosslinkers and antibodies after silanization.

FIG. 9K is an SEM image showing a close-up view of the bacteria bound to the biosensor.

FIG. 9L is an SEM image showing bacteria bridge electrodes together.

FIG. 10 shows an impedance spectra from 0 to 700,000 Ohms over a 10 Hz to 100 MHz frequency range for biosensors which had been immersed in solutions containing different concentrations of generic E. coli and then immersed in a neutral buffer for measuring the impedance.

FIG. 11 shows an impedance spectra from 0 to 10,000 Ohms over a 10 Hz to 100 MHz frequency range for biosensors which had been immersed in solutions containing different concentrations of generic E. coli and then immersed in a neutral buffer for measuring the impedance.

FIG. 12 shows a log-scale impedance spectra from 10 to 1,000,000 Ohms over a 10 Hz to 100 MHz frequency range for biosensors which had been immersed in solutions containing different concentrations of generic E. coli and then immersed in a neutral buffer for measuring the impedance.

FIG. 13 shows a phase impedance spectra over a frequency range of 10 Hz to 100 MHz for biosensors which had been immersed in solutions containing different concentrations of generic E. coli and then immersed in a neutral buffer for measuring the impedance.

FIG. 14 shows an impedance spectra over time for a biosensor which had been immersed in a solution containing 1×10⁶ CFU/mL of generic E. coli and then immersed in a neutral buffer for measuring the impedance.

FIG. 15 shows the phase over time from 100 Hz to 100 MHz for a biosensor which had been immersed in a solution containing 1×10⁶ CFU/mL of generic E. coli and then immersed in a neutral buffer for measuring the impedance.

FIG. 16 is a photomicrograph image of the interdigitated electrode array of a biosensor.

FIG. 17 is a photomicrograph image of the interdigitated electrode array and electrode traces of the biosensor in FIG. 16.

FIG. 18 is a photomicrograph of the electrode array of an unused biosensor.

FIG. 19 is a photomicrograph of the electrode array which shows the buildup of oxysilane on the surface after repeated use.

FIG. 20 shows the impedance spectra from 0 to 2,000 Ohms over a 10 Hz to 100 MHz frequency range for biosensors which had been immersed in solutions containing different concentrations of generic E. coli and then immersed in a neutral buffer for measuring the impedance.

FIGS. 21A and 21B are drawings of a scissors clamp 300 for the biosensor 10.

FIGS. 22A and 22B show the clamp 300 of FIGS. 21A and 21B mounted on a ring stand 301.

FIGS. 23A and 23B show the electrical contact with the biosensor 220 as shown in FIG. 3A.

FIG. 24 is a schematic diagram of the current 400 associated with the clamp 300 and biosensor 220.

FIGS. 25 to 32 are graphs showing test results with a non-pathogenic and a pathogenic E. Coli.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

As used herein, the term “capture reagent” includes any antibody capable of binding a specific analyte, any nucleic acid or nucleic acid analog capable of binding a specific nucleic acid analyte, any receptor capable of binding a specific analyte, any enzyme capable of binding a specific analyte (substrate),

As used herein, the term “antibody” or “antibodies” is intended to be a generic term which includes polyclonal antibody or antibodies, monoclonal antibody or antibodies, Fab fragment or fragments, single V_{H} chain antibody or antibodies such as those derived from a library of camel or llama antibodies or camelized antibodies (Nuttall et al., Curr. Pharm. Biotechnol. 1: 253-263 (2000); J. Biotechnol. 74: 277-302 (2001)), and recombinant antibody or antibodies.

As used herein, the term “recombinant antibody” or “recombinant antibodies” is a generic term which includes single polypeptide chains comprising the polypeptide sequence of a whole heavy chain antibody or only the amino terminal variable domain of the heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides) (See, Schmiedl et al., J. Immunol. Meth. 242: 101-114 (2000); Schultz et al., Cancer Res. 60: 6663-6669 (2000); Dubel et al., J. Immunol. Meth. 178: 201-209 (1995); and in U.S. Pat. No. 6,207,804 B1 to Huston et al.). Construction of recombinant single V_{H} chain or scFv polypeptides for a wide range of analytes can be obtained easily through currently available molecular techniques such as phage display (de Haard et al., J. Biol. Chem. 274: 18218-18230 (1999); Saviranta et al., Bioconjugate 9: 725-735 (1999); de Greeff et al., Infect. Immun. 68: 3949-3955 (2000)) or polypeptide synthesis. In further embodiments, the recombinant antibodies include modifications such as an attachment polypeptide having particular amino acid residues or ligands which facilitate binding of the recombinant antibody to the biosensor. Further still embodiments include fusion polypeptides which comprise the above polypeptides fused to a second polypeptide such as a polypeptide comprising protein A or G.

The present invention provides a device comprising a biosensor which combines a capture reagent method such as an antibody capture method with an impedance detection method. The biosensor is designed to detect analytes, in particular bacteria, viruses, toxins, parasites, and the like, in large sample volumes by measuring the increase in impedance induced by the analyte binding the capture reagent or antibody when the biosensor is introduced into the sample volume. Because of the ability to use the biosensor to probe large sample volumes, the biosensor enables detection of the analyte in a relatively short period of time, in general less than five minutes. Thus, the biosensor can provide real-time detection of an analyte in a large sample volume.

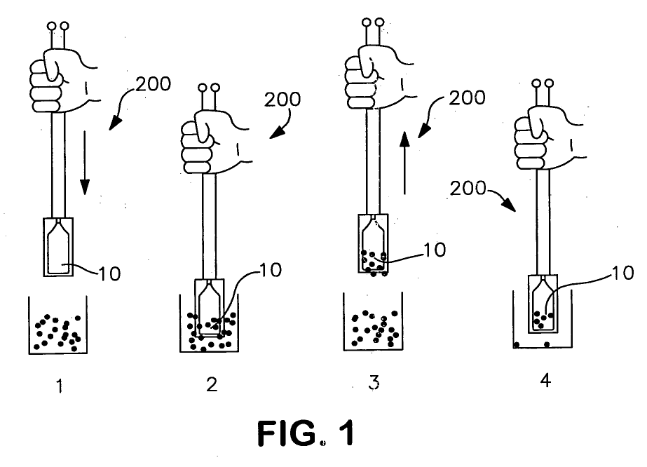

The present invention was developed for detection of bacteria in large bulk solutions. Hence, the dipstick-style sample procedure for its use. This is illustrated in FIG. 1. As shown in FIG. 1, the biosensor 10 is mounted on a support means to provide a handheld device 200 for detecting analytes, in particular bacteria, in a solution. The biosensor 10 comprising the handheld device 200 is simply inserted into a sample solution for several minutes (step 1), incubated in the solution for several minutes (step 2), removed from the solution (step 3), and then inserted into a neutral solution such as water and the impedance measured about two minutes thereafter (step 4). Thus, in about five minutes, the user can determine whether a large sample volume contains a particular species of bacteria. Unlike other devices, there are no tubes, valves, pumps, or enclosed cavities involved. All that is needed is for the biosensor to be dipped into the sample volume and then re-dipped into a clean, neutral solution such as water for analysis.

The present invention is distinguishable from biosensors currently being used to detect bacteria. The biosensor of the present invention detects the actual cell in contact with the electrode because the presence of bacteria on the sensor surface immediately causes a detectable impedance change. The unique combination of a micron-sized interdigitated electrode allows the bacteria (about 2 μm diameter) to land in between electrode gap. Thus, the biosensor of the present invention provides a real-time indication as to whether a sample contains a particular bacteria species. This is different from current biosensors where the conductivity of the bulk solution is measured to determine the presence of bacteria. Those technologies require the bacteria to grow in solution over several hours (or more), thereby increasing the ionic concentration of the bulk solution sufficiently for the biosensor to detect the resistance caused by the bacteria. Hence, they measure the change in resistance of the solution over time and are not measuring the concentration of bacteria that was actually in the sample prior to cultivation. Therefore, the current methods cannot provide a real-time indication as to whether a sample contains a particular bacteria species. The biosensor is further distinguishable in that it can detect bacteria suspended in bulk solutions. That means that large sample sizes such as those between 10 mL to 100 mL can be tested for bacteria contamination. In contrast, current biosensors are designed for measuring analytes in very small volumes. For example, much biosensor research has been focused on detecting enzymes in samples of blood. As a consequence, typical sample sizes for similar impedance-based biosensors range from 50 nL to 100 μL. As an another example, a microfluidic biosensor was developed by Purdue University in which a solution is passed over a set of interdigitated electrodes. The Purdue sensor, however, uses a nanoliter-sized sample which flows through an enclosed cavity.

Impedance spectroscopy is a technique used to measure the change in electrical impedance, Z, over a wide range of signal frequencies. The frequency range selected is 10⁰ Hz to 10⁷ Hz based on the 1-dispersion of the cell and the dielectric properties of cytoplasm and the lipid bilayer membrane (BLM) center in the 10⁰ Hz to 10⁷ Hz frequency range (Markx and Davey, (1999); Ciureanu et al., (1997)). Impedance analysis techniques can detect small changes in electrical current, on the order of 10⁻⁹ A, which can be translated into impedance, conductivity, resistance and capacitance (Tien, (2000)).

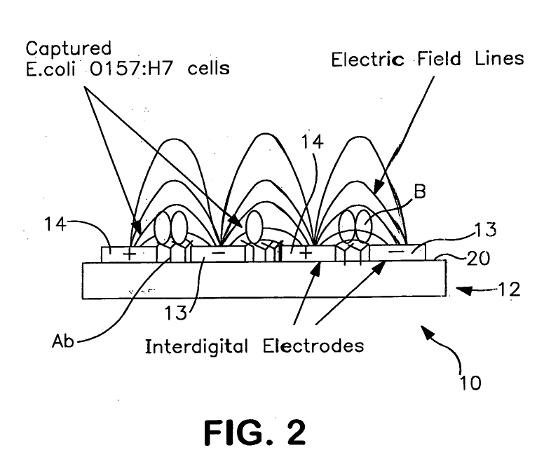

Detection theory of the present invention is illustrated in the cross-sectional view of the interdigitated electrodes 13, 14 of the biosensor 10 shown in FIG. 2 which uses antibodies as the capture reagent. Surface antibodies (Ab) are immobilized on the surface 20 of the support 12 between the interdigitated electrodes 13, 14 on the surface 20 by crosslinkers. The immobilized antibodies (Ab) act as nanotethers to hold the bacteria (B) in place. When the biosensor 10 is immersed in a sample solution containing bacteria, the bacteria (B) are bound by the immobilized antibodies (Ab). An electric circuit through the interdigitated electrodes 13, 14 induces an electric field (F) between the interdigitated electrodes 13, 14. The bound bacteria (B) cause a detectable change in the impedance in the electric field (F) between the electrodes 13, 14. The total impedance consists of a transient component (inductance and capacitance) and a non-transient component (resistance). As the signal frequency increases, the transient impedance decreases causing the impedance to be resistive between the electrodes 13, 14. Different cellular concentrations of bacteria (B) yield different changes in impedance between the electrodes 13, 14. Different genuses of bacteria can induce different changes in impedance over the frequency range. Therefore, the biosensor can distinguish between genuses of bacteria based upon the impedance produced over the frequency range.

The biosensor is fabricated by depositing an ultra-thin layer of metal onto the surface of a silicon chip to form an interdigitated electrode array and then covalently binding capture reagent or antibodies specific for a particular analyte onto the silicon surface between the interdigitated electrodes. The biosensor is then mounted on a support means such as an elongated probe or handle to provide the handheld device. The probe or handle enables the biosensor to be immersed into large sample volumes. Detection of an analyte is carried out by immersing the biosensor into a large sample volume for a time sufficient for the capture reagent or antibodies to bind the analyte, removing the biosensor from the sample volume, immersing the biosensor into an aqueous neutral solution, and measuring the change in impedance wherein an increase in impedance indicates that the sample contains the analyte.

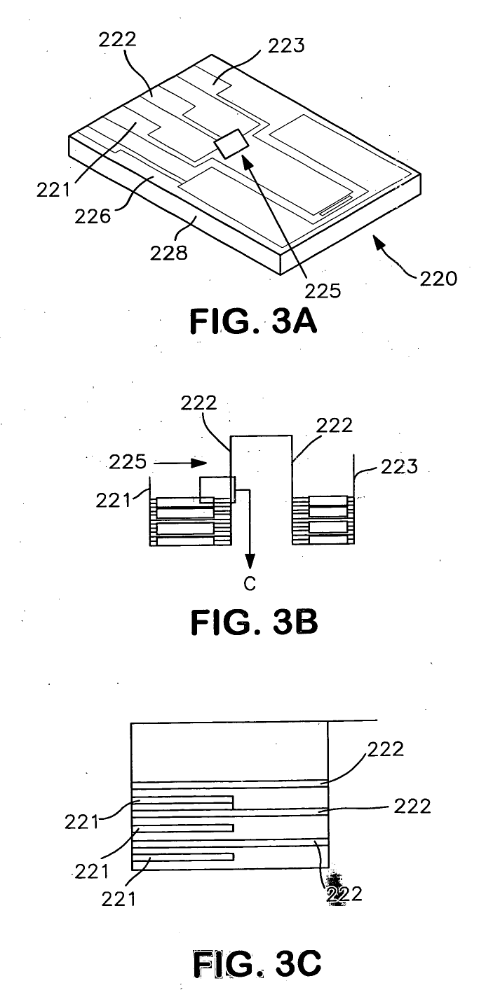

FIGS. 3A, 3B, and 3C illustrate one aspect of a biosensor 220 comprising the present invention which uses antibodies as the capture reagent. This aspect can use a capture reagent other than the antibodies shown. FIG. 3A shows central electrode 222 and lateral electrode 221 and 223 disposed on the surface 226 of support 228 and forming an interdigitated array 225. FIG. 3B shows a section of the interdigitated array 225 and shows the interdigitation of electrode 222 with electrodes 221 and 223. FIG. 3 shows a close-up of the interdigitated electrodes 221 and 222 of the interdigitated array 225. Electrodes 221 and 222, or 223 and 222, or all three electrodes can be used. The generator electrode can be electrode 222 and the collector electrode can be 221, 223, or both, or vice versa, the collector electrode can be electrode 222 and the generator electrode can be 221, 223, or both.

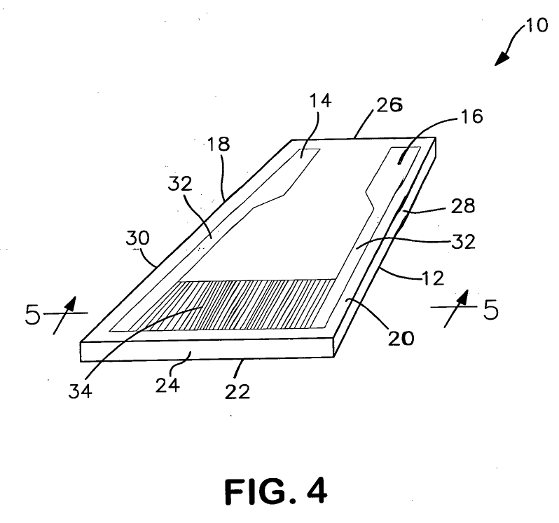

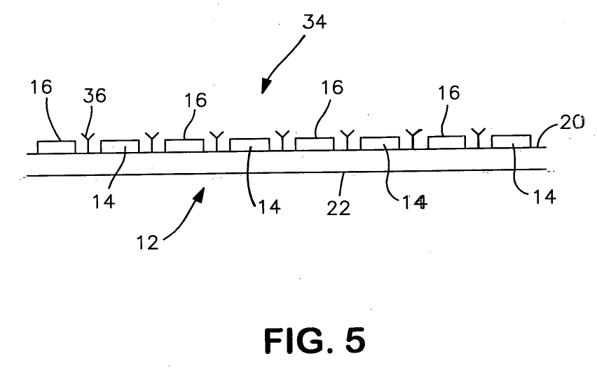

FIGS. 4 and 5 illustrate another aspect of the present invention which uses antibodies as the capture reagent. This aspect can use a capture reagent other than the antibodies shown. In this aspect, a biosensor 10 is provided having a support element 12 and electrically conductive tracks 14 and 16 disposed on thereon. The support element 12 comprises a body portion 18 which has a first surface 20 that supports conductive tracks 14 and 16 and an opposite surface 22. The body portion 18 has opposite ends 24 and 26 and edges 28 and 30 extending between ends 24 and 26. The body portion 18 can be any shape; however, in a preferred embodiment, the body portion 18 is rectangular in shape.

The support element 12 can be constructed from a wide variety of insulative materials. Non-limiting examples of insulative materials include glass, ceramic, vinyl polymers, polyimides, polyesters, and styrenics. In a preferred embodiment, the support element 12 comprises a silicon wafer, preferably a silicon wafer with 100 crystal orientation with an insulating layer thereon.

As shown in FIGS. 4 and 5, the electrically conductive tracks 14 and 16 are created on the first surface 20 of support element 12. Tracks 14 and 16 represent the electrode set of the biosensor 10. An electrode set includes at least two electrodes and may include any number of electrodes beyond two electrodes. For example, the electrode set can include a working and an auxiliary electrode or a generator electrode and a collector electrode. The electrode set can further include a reference electrode or a counter electrode or both. The electrode set can include any combination of the foregoing. As shown in FIG. 4, tracks 14 and 16 cooperate to form an interdigitated electrode array 30 on the first surface 20 of support element 12 and leads 32 that extend from array 34 across body portion 18 to end 26. Track 14 can be a working or generator electrode and track 16 can be an auxiliary or collector electrode.

Tracks 14 and 16 can be constructed of any electrically conductive material. Non-limiting examples include aluminum, carbon (for example graphite), cobalt, copper, gallium, gold, indium, iridium, iron, lead, magnesium, mercury, nickle, niobium, osmium, palladium, platinum, rhenium, rhodium, selenium, silicon, silver, tantalum, tin, titanium, tungsten, uranium, vanadium, zinc, zirconium, mixtures thereof, and alloys, oxides, or metallic compounds of these elements. In a preferred embodiment, the tracks 14 and 16 include a noble metal such as gold or an alloy thereof.

As shown in the cross-sectional view of the interdigitated array 34 (FIG. 5), the biosensor 10 further includes antibodies specific for a particular analyte 36 disposed on the first surface 20 of the body portion 18 between tracks 14 and 16 comprising the array 34.

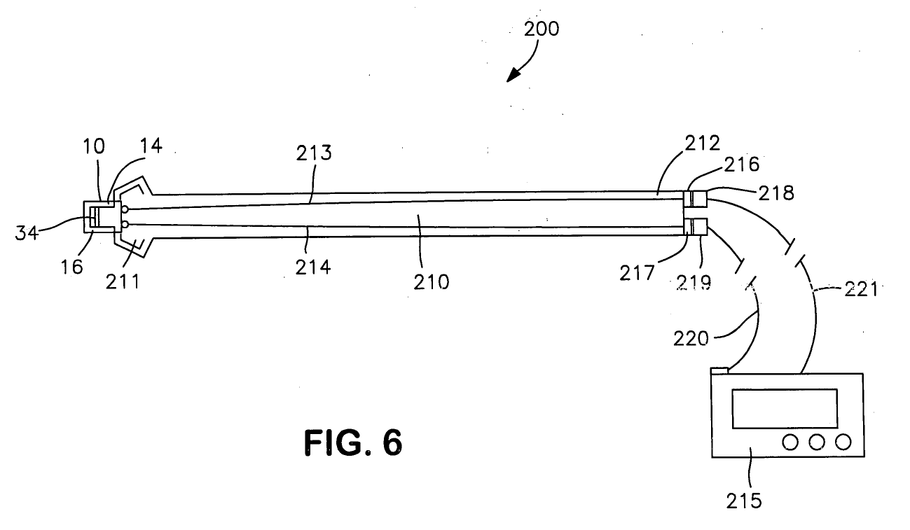

To enable facile detection of an analyte in large sample volumes, the biosensor of the present invention is mounted on a support means suitable to provide the handheld device of the present invention. The handheld device enables the biosensor to be immersed into a large sample volume. FIG. 6 shows an aspect of the present invention wherein the biosensor 10 is mounted on the distal end 211 of an elongated probe 210 which has a proximal end 212 for gripping to provide the handheld device 200. The elongated probe 210 enables gripping at the proximal end 212 by hand for inserting the biosensor 10 at the distal end 211 into a sample solution. The biosensor 10 is removably secured to the distal end 211 by a socket such as a USB connector adapted to receive and removably secure the biosensor 10 such that the electrode leads 14 and 16 are in contact with contact pads of the socket (not shown). The socket is preferably such that a biosensor 10 can easily be “plugged in” or “unplugged” from the elongated probe 210. The contact pads are operably connected via leads 213 and 214 to an impedance detector 215. Additional leads are included when the biosensor 10 includes auxiliary or reference electrodes, or both. In some embodiments, the impedance detector is an integral part of the proximal end 212 of the handheld device 200. In other embodiments, the leads 213 and 214 terminate at the proximal end 212 of the elongated probe 210 with connectors 216 and 217 which enable connectors 218 and 219 of leads 220 and 221 from an impedance detector 215 to be operably connected to the handheld device 200.

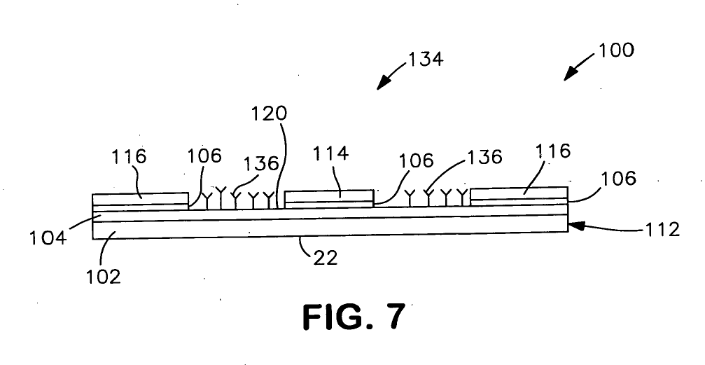

FIG. 7 shows a cross-sectional view of a biosensor 100 across interdigitated array 134 in accordance with a preferred embodiment of the present invention which uses antibodies as the capture reagent. This embodiment can use a capture reagent other than the antibodies shown. In the preferred embodiment, the support 112 of the biosensor 100 comprises a silicon wafer 102 with an insulator layer 104 passivated thereon which has surface 120. Preferably, the insulator layer 104 is in the form of silicon dioxide. The biosensor 100 further comprises an ultra-thin layer of metal deposited onto the photoresist layer 106 disposed on the surface 120 of insulator layer 104 to form electrically conductive tracks 114 and 116 which cooperate to form interdigitated array 134. In a preferred embodiment, the metal is a noble metal such as platinum, palladium, or gold, most preferably, gold. Disposed on the surface 120 of the insulator layer 104 are immobilized antibodies 136.

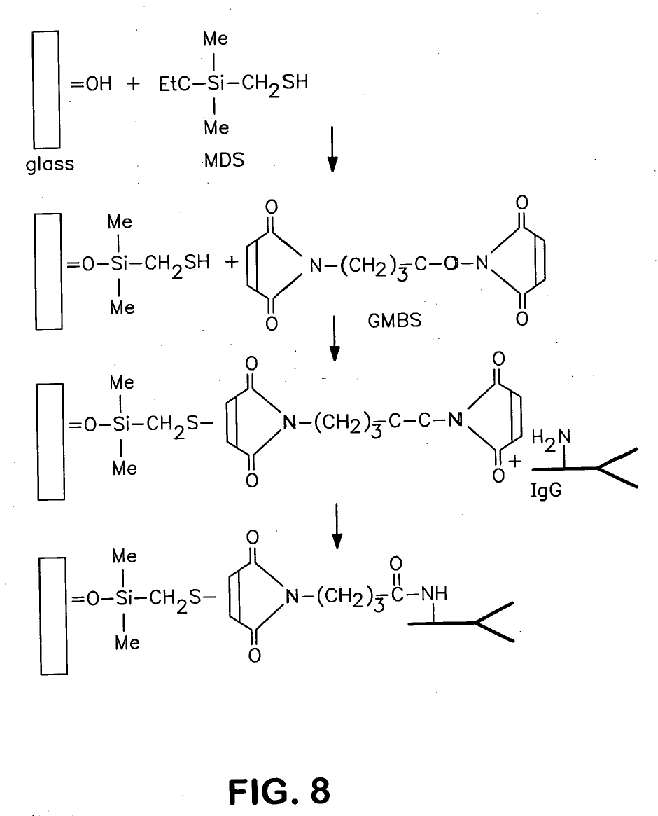

Attachment of antibodies to the biosensor surface is illustrated in FIG. 8 and described below. The biosensor surface is cleaned by immersing the biosensor chip into nitric acid for about 30 minutes. Afterwards, the chip is removed, washed with deionized water, and dried under a stream of nitrogen. Next, the chip is immersed in a mixture of hydrochloric acid and methanol (1:1) for about 30 minutes. Afterwards, the chip is removed, washed with deionized water, and dried under a stream of nitrogen. Next, the chip is immersed in sulfuric acid for about 30 minutes. Afterwards, the chip is removed, washed with deionized water, and dried under a stream of nitrogen. Next, the chip is immersed in a silanizing solution of about 2% 3-mercaptomethyl-dimethyl-ethoxysilane in dry toluene under a nitrogen atmosphere for about an hour. Afterwards, the chip is rinsed with dry toluene and dried under a stream of nitrogen. The silanized surface is then reacted with 2 mM N-γ-maleimidobutyryloxy succinimide ester (GMBS) dissolved in dimethylformamide (DMF) and ethanol at about 4° C. for about an hour. Then, after washing with phosphate buffered saline (PBS) or the like, the chemically modified surface is reacted with a solution containing antibodies (about 100 μg/mL in a saline solution such as PBS) at about 4° C. for about 24 hours. This produces a biosensor with antibodies immobilized to the biosensor surface between the interdigitated fingers of the electrodes. However, some antibodies may also immobilized to the surface of the electrodes as well. It is preferable that the biosensor chips be maintained in a moist environment at about 4° C. prior to use. To reuse the biosensor, the above cleaning process can be used to remove the antibodies or antibodies complexed with analyte from the biosensor surface.

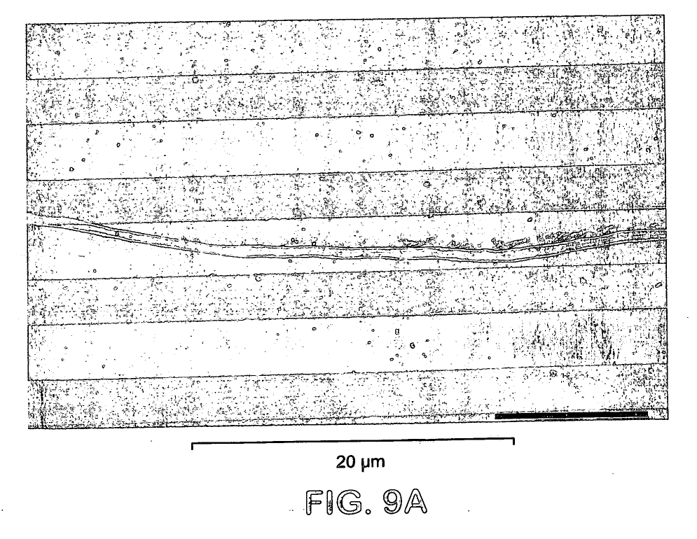

While FIGS. 2, 5, and 7 show antibodies immobilized solely on the surface of the support, in practice, the antibodies are also bound to the surface of the electrodes. The reason the antibodies bind to the surface of the electrodes is unclear; however, it is known that proteins can bind noble metals such as gold via sulfhydryl groups on the proteins. Other capture reagents, in particular, protein capture reagents, might also bind to both the surface of the support and the surface of the electrodes. Therefore, the biosensors include biosensors wherein the antibodies or other capture reagents are bound at least to the surface of the support which includes biosensors in which the antibodies or other capture reagents are bound both to the surface of the support via crosslinking and to the surface of the electrodes via sulfhydryl groups on the protein. Antibodies bound to both the surface of the support and the surface of the electrodes can be seen in the series of scanning electron microphotographs (SEM) shown in FIGS. 9A to 9L.

FIG. 9A shows an SEM of gold electrodes (narrow light bands) on the silicon oxide surface (dark bands) of a silicon chip biosensor at a magnification of 4000×. FIG. 9B shows an SEM of gold electrodes on the silicon oxide surface after cross-linking antibodies to the silicon oxide surface as described above. As can be seen, the antibodies are bound both the surface of the gold electrodes and to the silicon oxide surface. FIGS. 9C and 9D show SEMs of the biosensor after being testing in a buffer solution containing a concentration of 10² CFU/mL of generic E. coli or 10⁶ CFU/mL of generic E. coli, respectively. The Figures show the bacteria bound by the antibodies. FIG. 9D shows a big ridge of bound bacteria spanning several electrodes.

FIG. 9E shows an SEM of gold electrodes (narrow light bands) on the silicon oxide surface (dark bands) of a silicon chip biosensor at a higher magnification of 8500×. FIG. 9F shows an SEM of gold electrodes on the silicon oxide surface after cross-linking antibodies to the silicon oxide surface as described above at the higher magnification. As can be seen, the antibodies are bound both the surface of the gold electrodes and to the silicon oxide surface. FIGS. 9G and 9H show SEMs of the biosensor after being testing in a buffer solution containing a concentration of 10² CFU/mL of generic E. coli or 10⁶ CFU/mL of generic E. coli, respectively. The Figures show the bacteria bound by the antibodies. FIG. 9H shows bound bacteria bridging several electrodes.

FIG. 9I shows an SEM of gold electrodes (narrow light bands) on the silicon oxide surface (dark bands) of a silicon chip biosensor at an even higher magnification of 40,000×. FIG. 9J shows an SEM of gold electrodes on the silicon oxide surface after cross-linking antibodies to the silicon oxide surface as described above at the higher magnification. As can be seen, the antibodies are bound both the surface of the gold electrodes and to the silicon oxide surface. FIGS. 9K and 9L show SEMs of the biosensor after being testing in a buffer solution containing generic E. coli. The Figures show the bacteria bound by the antibodies. FIG. 9L shows a bound bacteria bridging two electrodes.

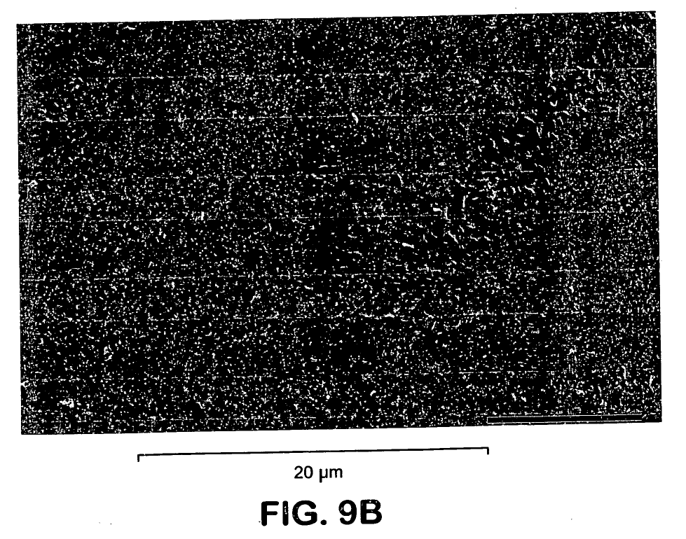

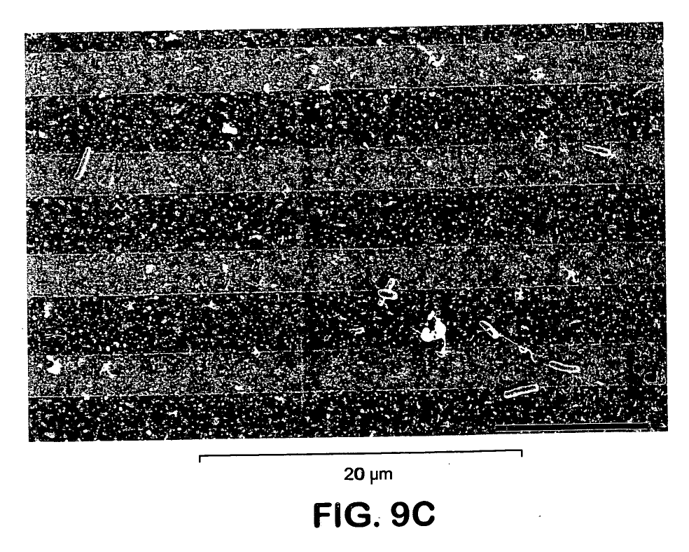

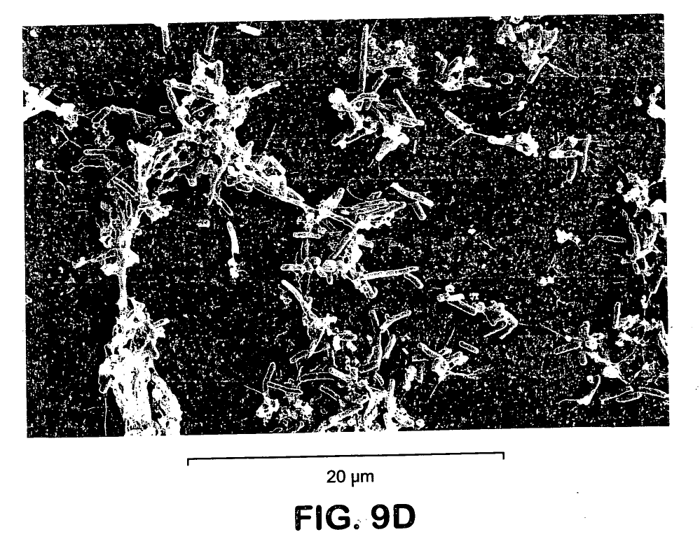

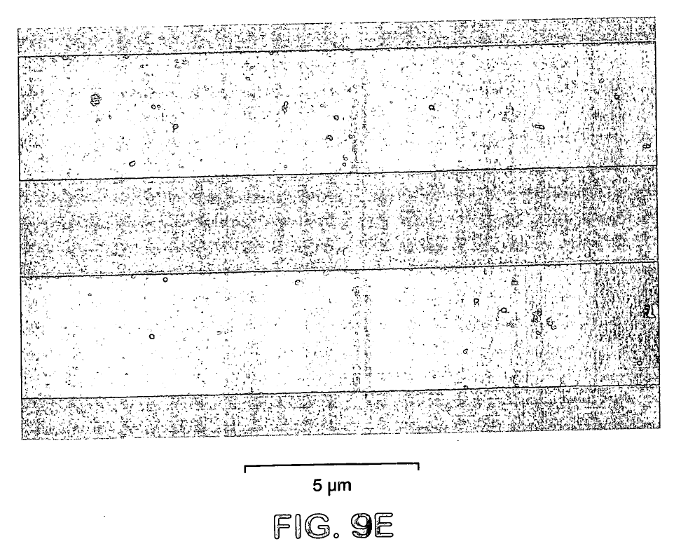

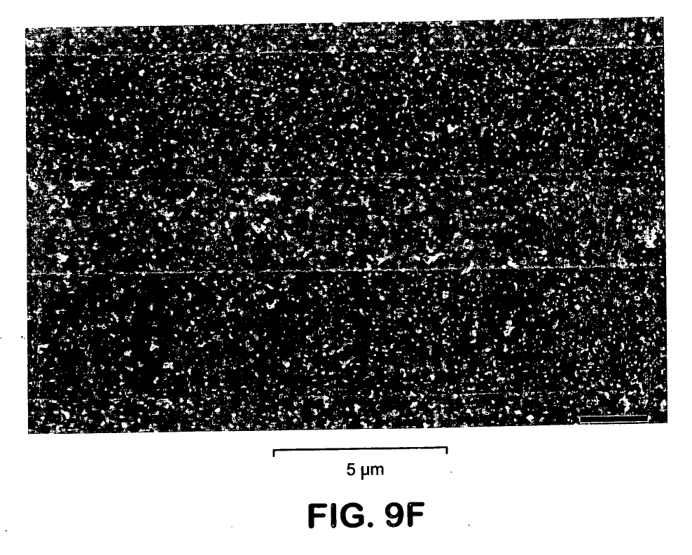

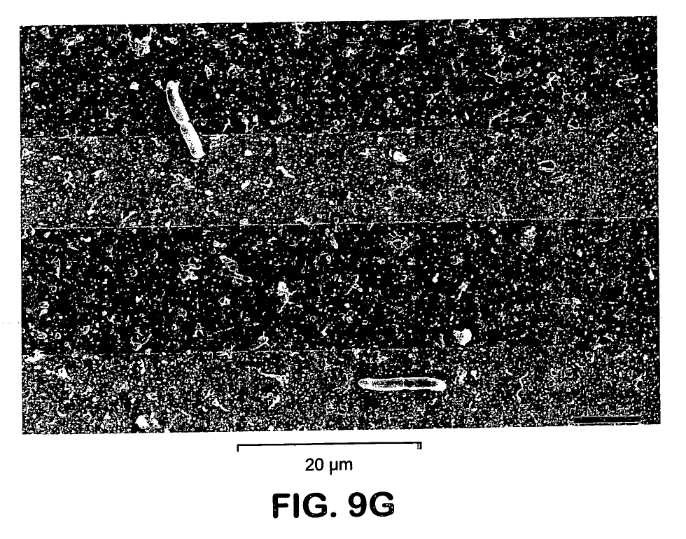

FIGS. 10 to 15 show examples of impedance spectroscopy data obtained with the biosensor inserted into samples containing various concentrations of E. coli. The total impedance was measured from a frequency range of 10 Hz to 100 MHz and plotted for the different concentrations shown. The measurements were all taken within five minutes of inserting the biosensor into spiked samples of E. coli in a pure culture. The slope of the impedance curves depends on the concentration of bacteria immobilized on the surface of the biosensor.

The handheld device of the present invention shows great promise for the detection, enumeration, and identification of bacterial pathogens found in the food and water supply and other biological matrices. The small feature size of the electrodes of the biosensor comprising the handheld device enables measurement of the events taking place at the cellular level. Applications of the handheld device of the present invention can be expanded to cover fresh fruits and vegetables, meats, dairy products and other foods. Applications of the handheld device can further include embodiments which use capture reagents other than antibodies. For example, embodiments wherein the capture reagent is a nucleic acids (DNA or RNA or analog thereof), enzymes, or natural or synthetic receptor.

The handheld device of the present invention can be constructed to comprise a biosensor which can detect in a bulk solution a pathogenic bacterium such as Salmonella spp., Clostridium botulinum, Staphylococcus aureus, Campylobacter jejuni, Yersinia enterocolitica and Yersinia pseudotuberculosis, Listeria monocytogenes, Vibrio cholerae O1, Vibrio cholerae non-O1, Vibrio parahaemolyticus and other vibrios, Vibrio vulnificus, Clostridium perfringens, Bacillus cereus, Aeromonas hydrophila and other spp., Plesiomonas shigelloides, Shigella spp., Miscellaneous enterics, Streptococcus, Escherichia coli strains such as enterotoxigenic (ETEC), enteropathogenic (EPEC), O157:H7 enterohemorrhagic (EHEC), and enteroinvasive (EIEC), or the like.

The handheld device of the present invention can be constructed to comprise a biosensor which can detect in a bulk solutions a pathogenic virus such as Hepatitis A virus, Hepatitis E virus, Rotavirus, Norwalk virus group, or the like.

The device of the present invention can be constructed to comprise a biosensor which can detect in a bulk solution a parasitic protozoa or worm such as Giardia lamblia, Entamoeba histolytica, Cryptosporidium parvum, Cyclospora cayetanensis, Anisakis sp. and related worms, Diphyllobothrium spp., Nanophyetus spp., Eustrongylides sp., Acanthamoeba and other free-living amoebae, Ascaris lumbricoides, Trichuris trichiura, or the like.

The handheld device of the present invention can be constructed to comprise a biosensor which can detect in a bulk solution a natural toxin such as ciguatera poisoning, shellfish toxins (PSP, DSP, NSP, ASP), scombroid poisoning, tetrodotoxin (Pufferfish), mushroom toxins, aflatoxins, pyrrolizidine alkaloids, phytohaemagglutinin (red kidney bean poisoning), grayanotoxin (honey intoxication), or the like

The handheld device of the present invention can also be constructed to comprise a biosensor which can detect in a bulk solution prions.

The following examples are intended to promote a further understanding of the present invention.

### EXAMPLE 1

A biosensor is constructed as follows. A 4 inch (100 mm) diameter silicon wafer with 100 crystal orientation serves as the foundation. Then 2 μm of silicon oxide is grown over the silicon to act as an insulator. Next, photoresist (PR) is spun onto the wafer which is then exposed to UV radiation through a photomask containing the electrode pattern. Afterwards, the PR is developed in the pattern of the photomask.

Next, 30 nm of titanium followed by 50 nm of gold is deposited onto the wafer to form an interdigitated electrode structure, followed by removal of the PR. The wafer is then diced into 8 mm×12 mm chips.

Antibodies are attached to the silicon surface of the chips as follows. The silicon surface is cleaned by immersing the chip into a mixture of hydrochloric acid and methanol for 30 minutes, and then in sulfuric acid for 30 minutes, followed by rinsing in distilled H₂O. The chip is then immersed in a silanizing solution of 3-mercaptomethyldimethylethoxysilane (MDS) and dry toluene.

Afterwards, the chip is immersed in a solution of dimethylformamide (DMF) and ethanol containing the crosslinker N-γ-maleimidobutyryloxy succinimide ester (GMBS). After the cross-linker has been attached to the silanized surface of the chip, antibodies are attached via the crosslinker to produce the biosensor in which the antibodies are immobilized to the surface between the electrodes.

### EXAMPLE 2

A prototype apparatus comprising a biosensor was fabricated and tested for ability to detect E. coli O157:H7 in a large sample volume.

The biosensor was fabricated from a 100 Silicon wafer with a 2 μm layer of SiO₂ as an insulating layer. The biosensor active area contained interdigital gold electrodes deposited over the SiO₂ using photolithographic processing methods. Analyte specific antibodies were immobilized to the SiO₂ in between the electrodes creating a biological sensing surface. Using impedance spectroscopy, the impedance across the interdigital electrodes was measured after immersing the biosensor in solution. Bacteria cells present in solution attached to antibodies and became tethered to the biosensor surface. Immobilized bacteria cells changed the dielectric constant and thus the capacitance, and the resistance of the media between the electrodes thereby causing a change in measured impedance. The biosensor was able to discriminate between different cellular concentrations from 10⁵-10⁷ CFU/mL in pure culture with a detection time of 5 minutes. The design, modeling, fabrication and testing of the biosensor is described below.

Fabrication of the Biosensor

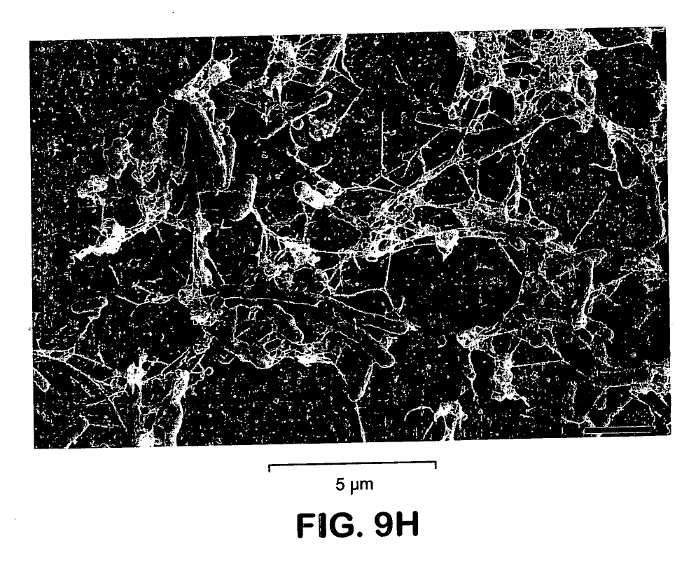

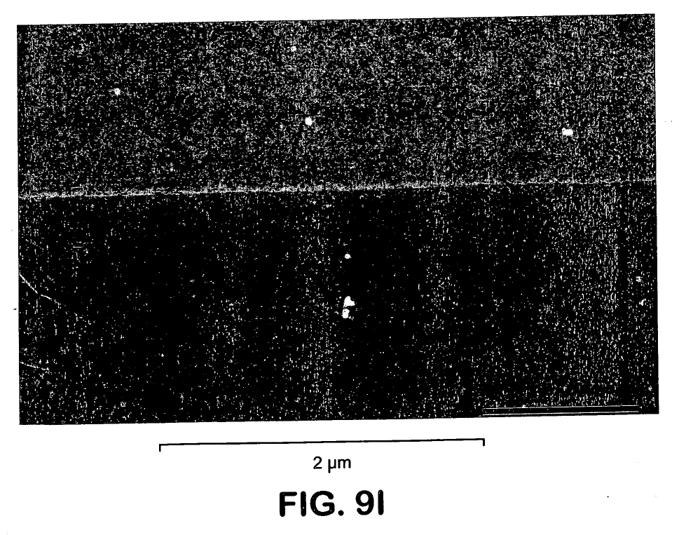

The biosensor was fabricated from 4″ (102 mm) 100 Silicon with a 2 μm thick layer of silicon dioxide. The oxide layer serves as an insulator between the electrodes and the silicon substrate. The electrodes were fabricated using a “lift-off” process. Briefly, photolithography was used to pattern photoresist in the pattern of the electrodes onto the oxide surface. The photoresist was spun on the wafer, exposed to UV light and developed. Metal was evaporated onto the wafer in a two-step process; a 3 nm layer of titanium was deposited for adhesion, followed by a 30 nm layer of gold for use as the conducting surface. “Lift-off” of the metal involved sonication of the wafer in photoresist developer solution. Metal deposited over the photoresist was removed (lift-off) to reveal the electrode pattern of the biosensor. After metal deposition, the wafer was diced into small individual sensor chips. The device includes an interdigitated electrode array (FIG. 16) with circuit traces connecting to bond pads (FIG. 17). The electrode geometry consists of 2 arrays of interdigital electrodes. Each electrode has a length of 0.8 mm and a width of 3 μm with an in-between spacing of 4 μm. The sensor has a large active area totaling 10 mm² when both arrays are included.

The next step in the fabrication of the biosensor was to immobilize the biological substrates to the oxide surface. The antibodies were attached to the surface via heterobifunctional crosslinkers using an established process (Bhatia et al., Anal. Biochem. 178: 408-413 1988). Briefly, the biosensor was cleaned and activated by immersing the chip into a mixture of hydrochloric acid and methanol for 30 minutes followed with immersion in sulfuric acid. The silanizing of 3-Mercaptomethyldimethylethoxysilane (MDS) and chip surface occurred in dry toluene. The crosslinker was attached to the glass surface after silanization. The crosslinker used was N-γ-maleimidobutyryloxy succinimide ester (GMBS) dissolved in dimethylformamide (DMF) and ethanol. The crosslinkers attached to the antibodies were added to the sensor surface.

After immobilizing the antibodies to the sensor surface, the biosensor was ready for testing. The sensor was placed in a test fixture where the bond pads were connected to leads terminating to a breadboard (and then to the impedance analyzer). The prototype device was complete and ready for testing.

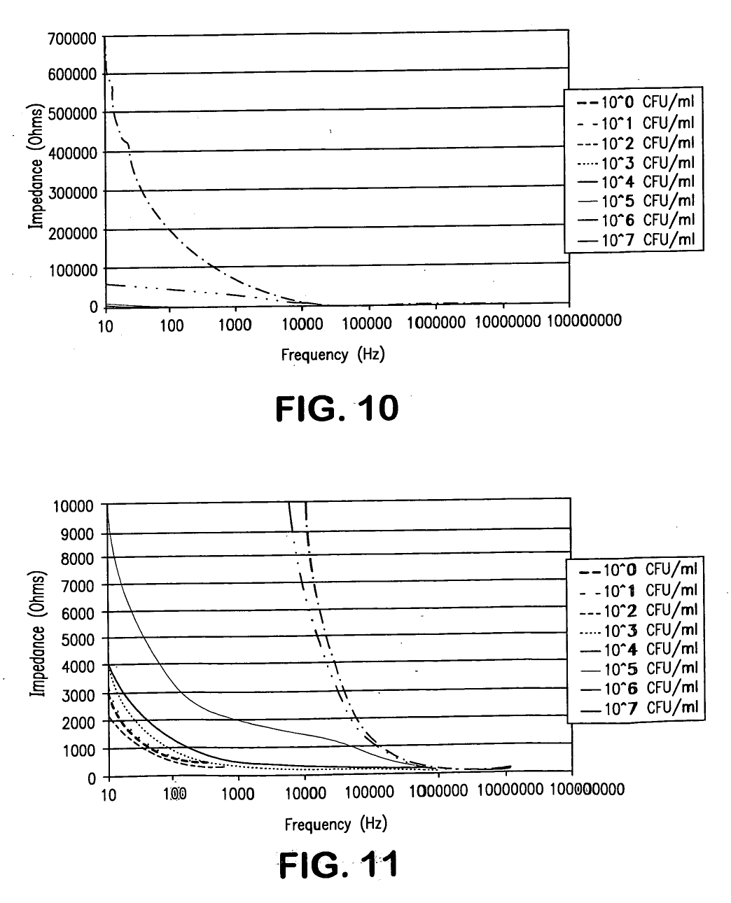

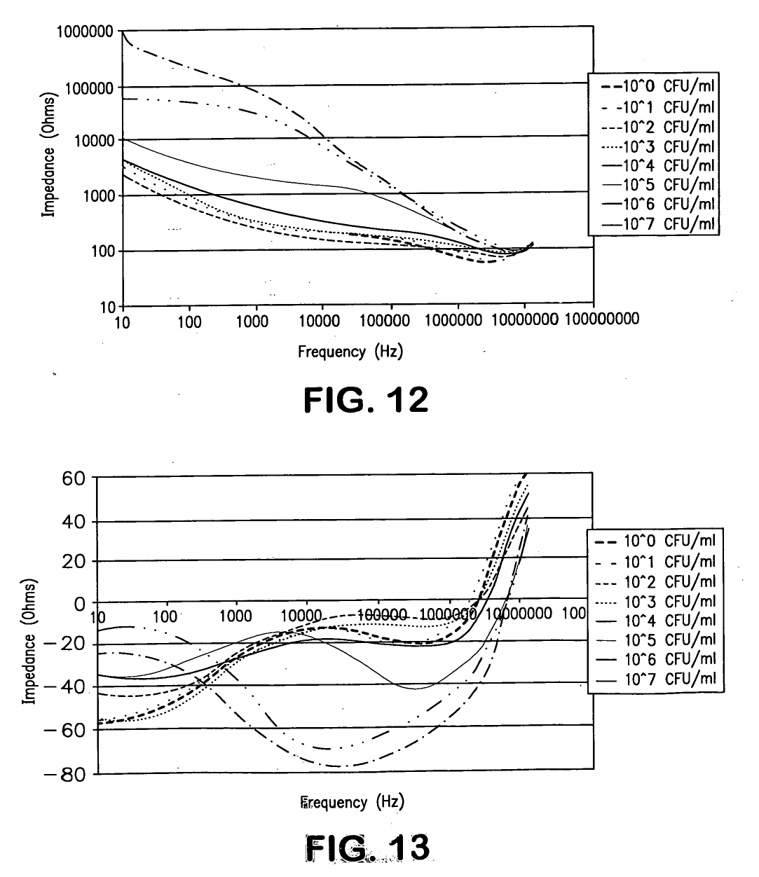

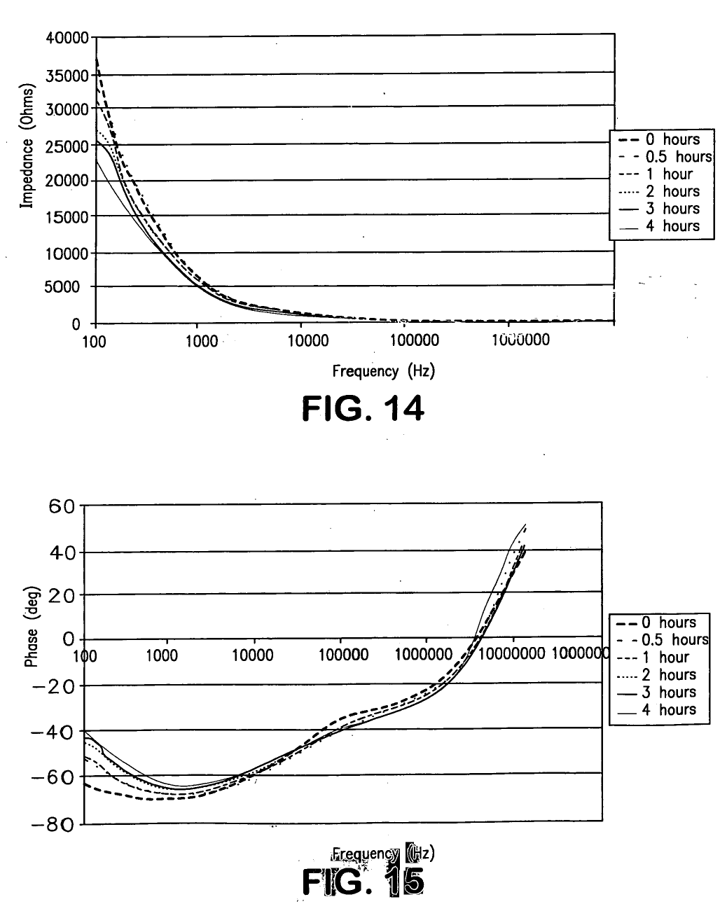

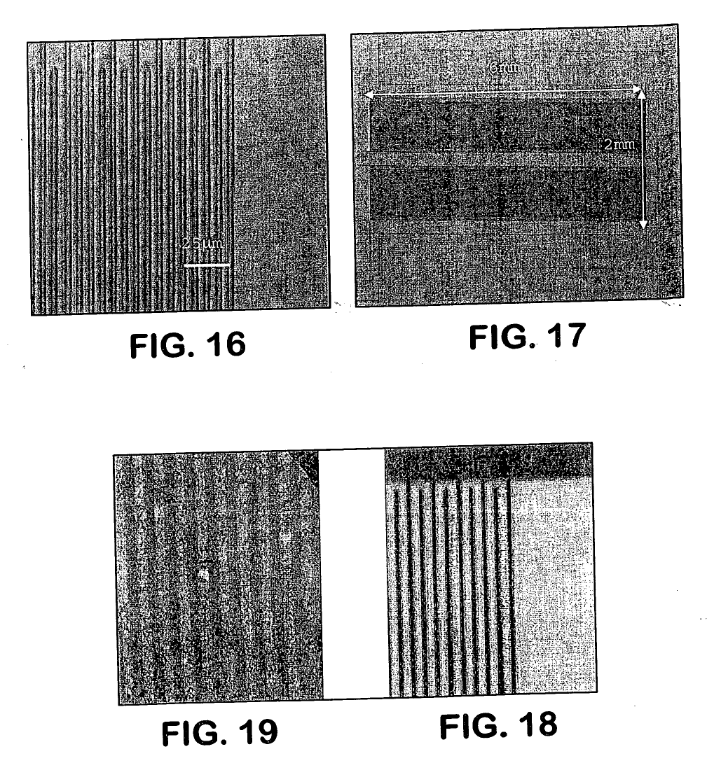

FIGS. 21A and 21B show a scissors clamp 300 holding the electrode leads of the biosensor 220 of the type shown in FIG. 3A. An electrical lead 301 leading to a 4-terminal electrical contact plate 302 (USB type) with insulating chucks 303A and 303B between jaws of the clamp 301. The lower chuck 303B locates the chip 220 in position. (See FIGS. 23A and 23B for drawings of the clamp and its four contacts.). A biosensor is inserted between the PVC chuck 303B and the USB plate 302 such that the PVC contacts the backside of the biosensor 220 which maintains contact between the electrodes 304 of the biosensor and the contacts of the USB plate 302. The plate 304 has three of its electrodes 304 in contact with electrodes 221, 222, and 223 of the biosensor 220 of the type illustrated in FIG. 3A. The electrodes 304 of the plate 302 in contact with the electrodes of the biosensor are connected via lead 301 to the terminals of a breadboard (not shown) which are then connected via a second set of leads to an impedance analyzer. FIGS. 22A and 22B show the clamp 300 mounted on a ring stand 305. The clamp 300 is moved into the solution for testing.

Testing

Test samples were prepared from varying concentrations of a stock culture of E. coli inoculated in buffer solution. The buffer was serially diluted into samples of 20 mL with concentrations ranging from 10⁰ CFU/mL to 10⁷ CFU/mL. The biosensor was immersed into the sample for 2 minutes to allow the antibody-antigen reaction to occur and form a complex with the analyte and the impedance was measured by an HP 4192A Impedance Analyzer. The entire process including data acquisition required 5 minutes. After measurement, the sample concentrations were determined by the standard plating method according to the FDA Bacteriological Analytical Manual (FDA, Bacteriological Analytical Manual, AOAC International, Gaithersburg, Md. (1998)).

Results and Discussion

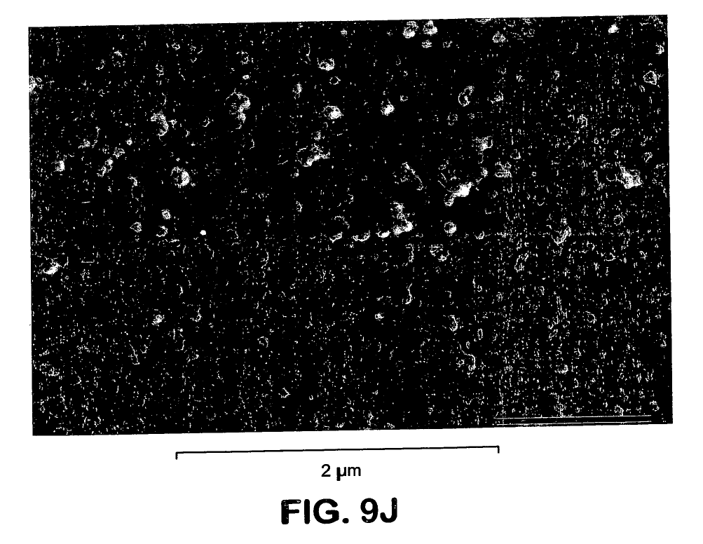

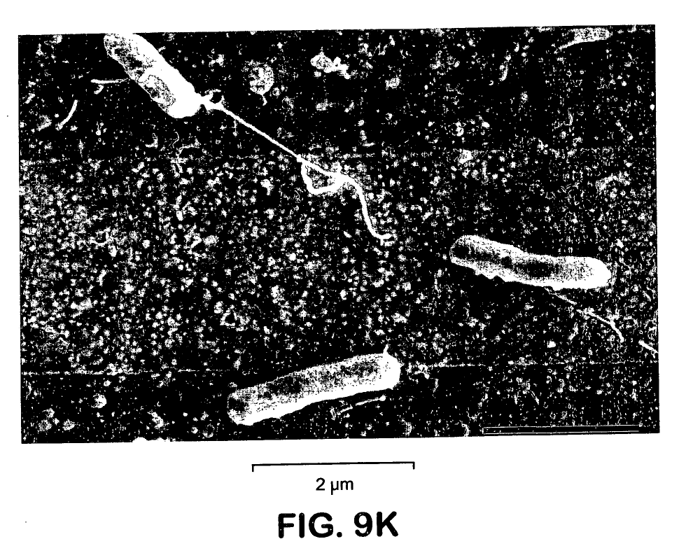

Multiple testing of the prototype biosensor caused a thin film of silanes to build up on the sensor surface (FIG. 19) compared to an unused sensor (FIG. 18). Though each sensor was cleaned thoroughly before testing of different cellular concentrations, the repeated treatment of the surface with oxysilanes changed the dielectric constant of the sensor surface between trials. Further experimentation will require the use of a new biosensor for each new test.

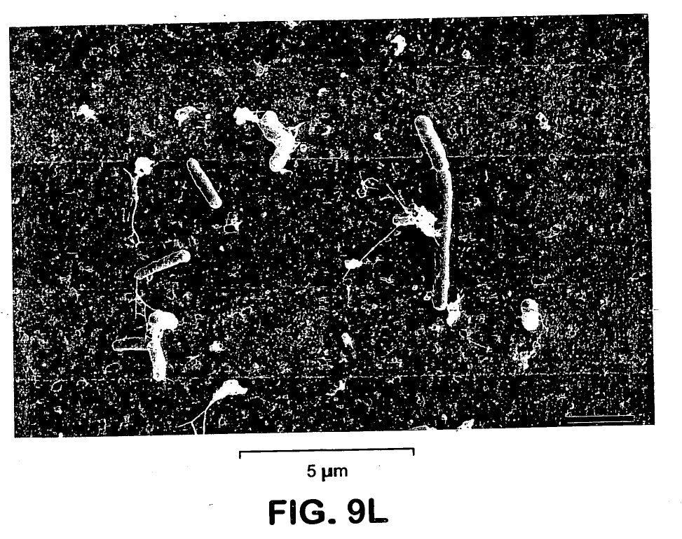

Even though there was damage to the sensor surface, the results of pure culture testing were promising. All samples showed the same trend of decreasing impedance with increasing frequency. At low frequencies, the impedance is dominated by the reactance of the membrane capacitance, while at high frequencies the impedance converges towards the resistance of the bulk solution (FIG. 20). Testing with a sterile solution yielded the lowest impedance value and is taken as the baseline measurement. With cells attached to the biosensor, the impedance generated was significantly higher due to the high resistivity of the cell's lipid bilayer. The concentration of the antigen influenced the impedance across the interdigitated electrodes.

Different bacteria concentrations are distinguishable by their impedance curve. The sensor behaves as expected, increasing impedance with an increase in cellular concentration, but not in a linear fashion. The non-linear behavior is suspected to occur for a combination of two reasons. First, as the concentration increases, the number of antibody binding sites was reduced and became saturated at a critical concentration. After all the antibody binding sites are filled, additional cells in solution do not bind to the sensor surface but stay suspended in solution changing the bulk resistance value. Second, the build up of oxysilanes on the sensor surface biased the results as more samples were tested. Further analysis of these trends will be continued after new biosensors are fabricated.

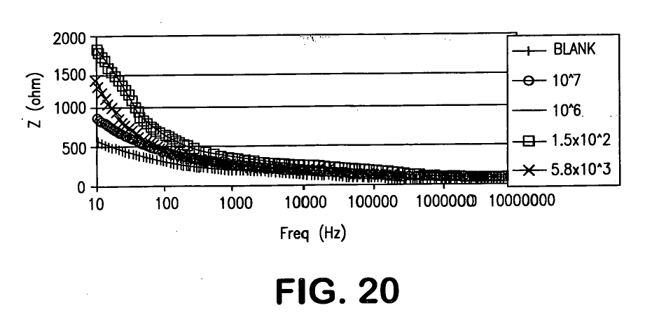

FIG. 25 shows the impedance characteristics for different concentrations of non-pathogenic E. coli in a pure culture solution. The impedance is shown to decrease with respect to the bacteria concentration present in the sample. For example, at a frequency of 1 kHz (x-axis) the highest impedance value is for a solution with a concentration of 10⁷ CFU/mL, the lowest impedance is for 10³ CFU/mL. Concentrations below 10³ CFU/mL (10², 10¹, 10⁰ and the control) are not shown since their impedance values are similar to that of 103 CFU/mL. The frequency range tested was 10 Hz-10 MHz. The optimum frequency for measuring impedance was found to be 1 kHz, since at lower frequencies the signal was unstable and at high frequencies the impedance of all bacteria concentrations converged.

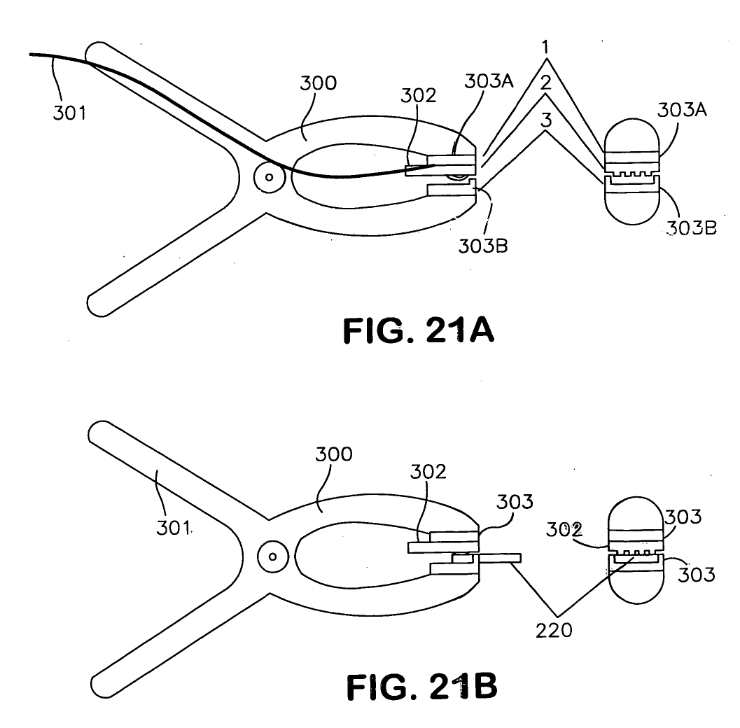

FIG. 26 shows the impedance characteristics for different concentrations of pathogenic E. coli O157:H7 in a pure culture solution. The impedance is shown to decrease with respect to the bacteria concentration present in the sample. For example, at a frequency of 1 kHz (x-axis) the highest impedance value is for a solution with a concentration of 10⁷ CFU/mL, the lowest impedance is for 10³ CFU/mL. Concentrations below 10³ CFU/mL (10², 10¹, 10⁰ and the control) are not shown since their impedance values are similar to that of 10³ CFU/mL. The frequency range tested was 10 Hz-10 MHz. The optimum frequency for measuring impedance was found to be 1 kHz, since at lower frequencies the signal was unstable and at high frequencies the impedance of all bacteria concentrations converged.

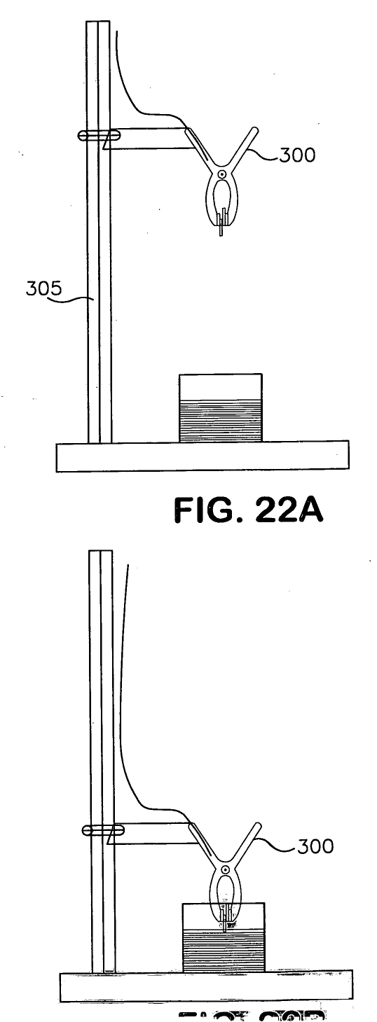

FIG. 27 shows the difference in impedance for E. coli and E. coli O157:H7 at a test frequency of 1 kHz. The figure shows that the impedance increases as bacteria concentration increases. This allows the biosensor to discriminate between high and low concentrations of bacteria solutions. It should also be noted that the impedances of low concentrations (10³, 10², 10¹, 10⁰ and the control) are similar and cannot be differentiated by the biosensor.

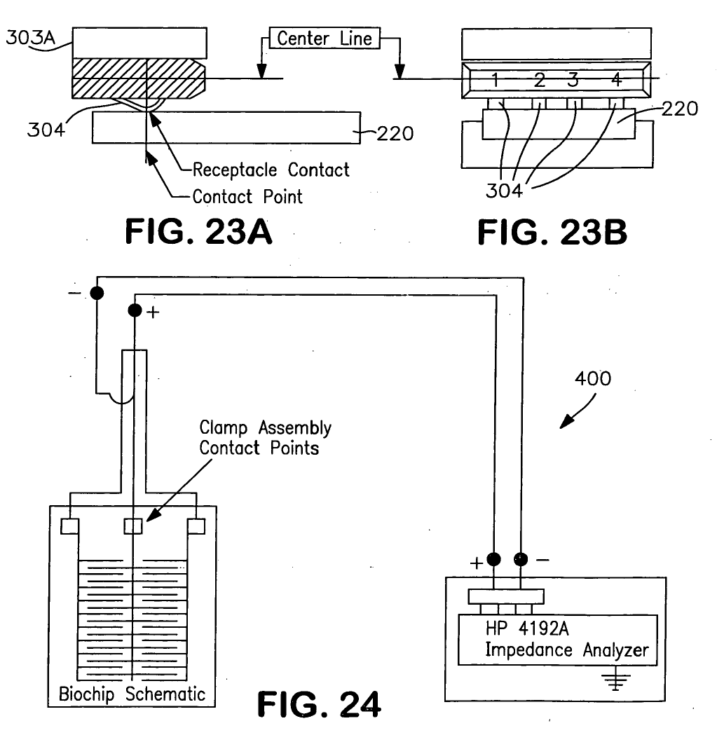

FIG. 28 shows the difference in impedance for E. coli and E. coli O157:H7 at a test frequency of 100 kHz. The figure shows that the impedance increases as bacteria concentration increases. This allows the biosensor to discriminate between high and low concentrations of bacteria solutions, but not as well as in FIG. 3 where the test frequency was 1 kHz.

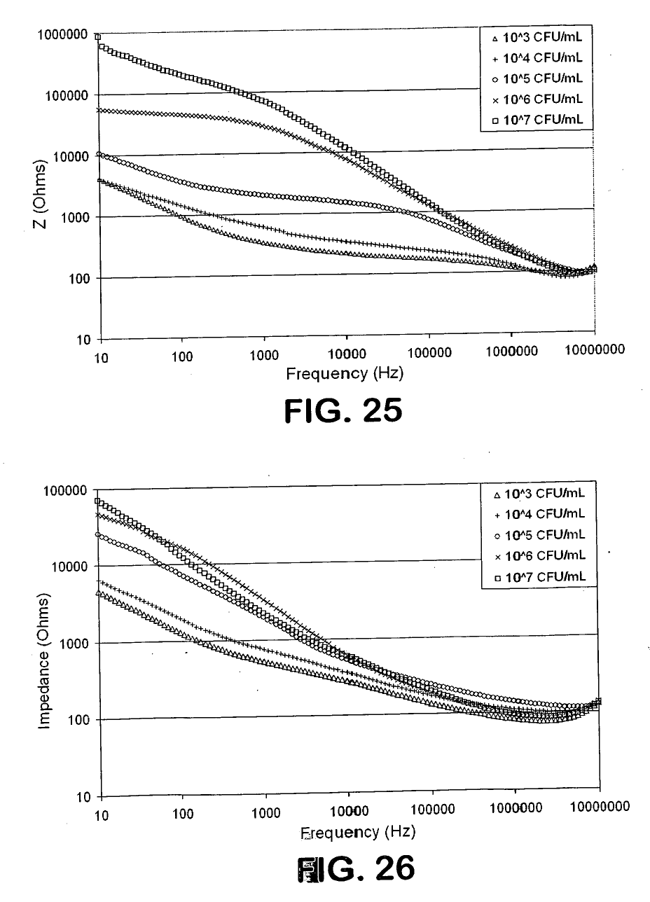

FIG. 29 shows the difference in impedance for E. coli and E. coli O157:H7 at a test frequency of 10 MHz. The figure shows that the impedance does not change as bacteria concentration increases. The figure shows that at high frequencies the impedance of all concentrations of bacteria solution are indistinguishable. (At high frequencies, the impedance properties of the bacteria have little effect on the biosensor and the dielectric properties of the testing solution become the dominant portion of total impedance.)

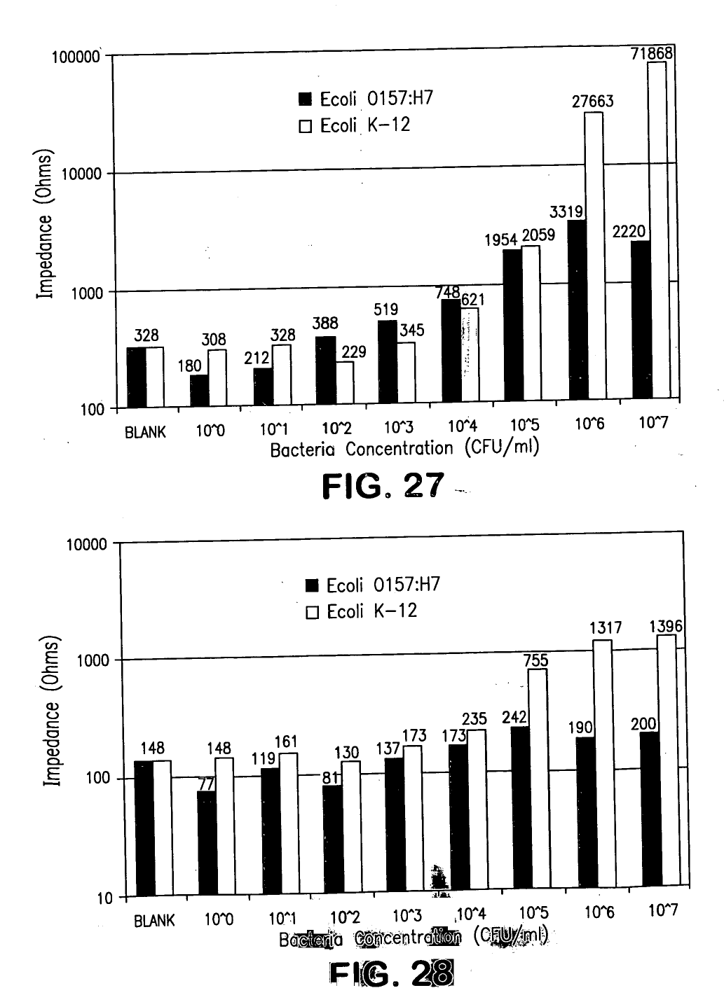

FIG. 30 shows the effects of foreign bacteria (Salmonella infantis) in the biosensor performance. Clearly, in the presence of only S. infantis, the biosensor cannot distinguish between different bacteria concentrations of S. infantis. In mixed cultures of E. coli O157:H7 and S. infantis, the impedance is shown to increase at high concentrations, thus demonstrating specificity to E. coli O157:H7 in the presence of other organisms.

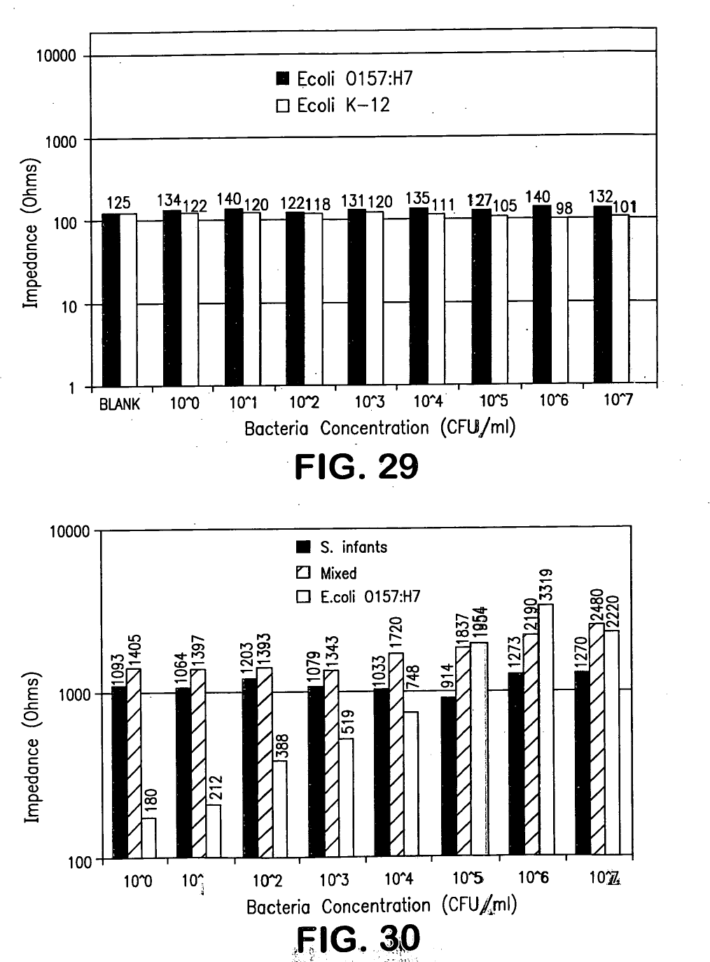

FIG. 31 shows the results of an analysis of variance followed by a Tukey's T test to determine the significance between the means of the impedance of non-pathogenic E. coli. (Impedance values are shown in log scale and are taken from FIG. 3.) It is shown that the biosensor has a statistically significant lower detection limit of 10⁵ CFU/mL for non-pathogenic E. coli in pure culture. This establishes that the biosensor can detect the difference between concentrations at 10⁵ CFU/mL or greater with respect to a sterile blank sample.

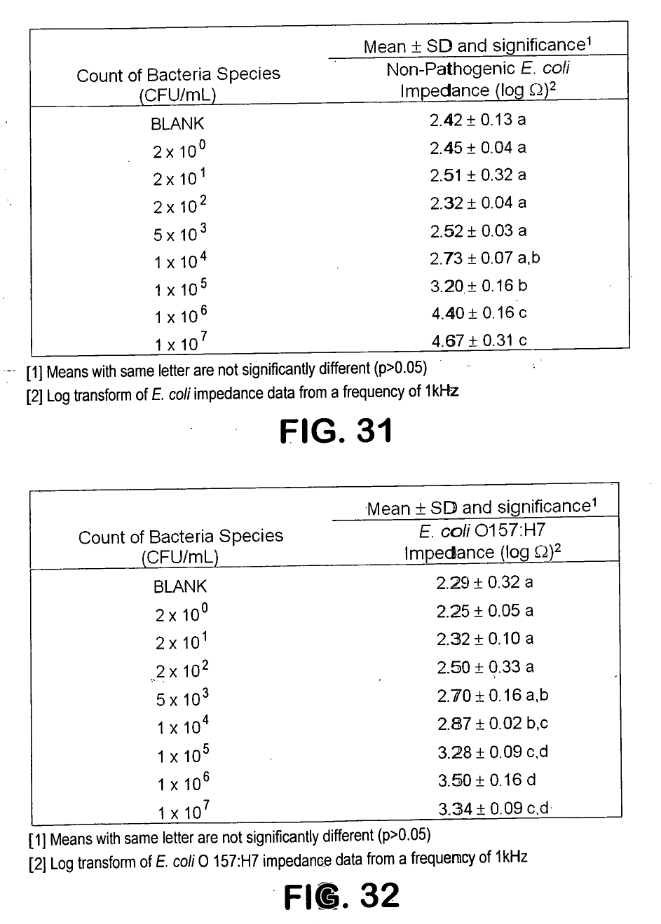

FIG. 32 shows the results of an analysis of variance followed by a Tukey's T test to determine the significance between the means of the impedance of E. coli O157:H7. (Impedance values are shown in log scale and are taken from FIG. 3.) It is shown that the biosensor has a statistically significant lower detection limit of 10⁴ CFU/mL for pathogenic E. coli O157:H7 in pure culture. This establishes that the biosensor can detect the difference between concentrations at 10⁴ CFU/mL or greater with respect to a sterile blank sample in a pure culture solution.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A device for detecting a microorganism which comprises:
(a) a holder means with an electrode means;
(b) a detection sensor mounted on the holder means in contact with the electrical means, the sensor having spaced apart interdigitated electrodes providing a capacitor and with a capture reagent for the microorganism at least between the electrodes, wherein widths of a space between the electrodes and of the electrodes are each about 5 microns or less, and wherein when the microorganism in a liquid is captured by the capture reagent, the detection sensor is interrogated with an electric circuit producing a change of impedance from the detection sensor without the bound microorganism, thereby detecting the presence of the microorganism.

2. The device of claim 1 wherein the electrode means are connected to a circuit for measuring the impedance.

3. The device of claim 2 wherein the device is adapted to be removed from the liquid after the microorganisms are bound to the detection sensor and then to be connected to the circuit for the interrogation of the sensor.

4. The device of any one of claims 1, 2 or wherein with a reference sensor is provided, adjacent 3 the detection sensor, without the capture reagent for detecting the change of impedance.

5. The device of any one of claims 1, 2 or 3 wherein the electrodes have a 4 μm (4000 nm) spacing and a 3 μm (3000 nm) width, and are provided on a non-conductive substrate.

6. The device of any one of claims 1, 2 or 3 wherein the captive reagent is specific for at least a genus classification of the microorganism and the microorganism is identified at least as to the genus classification.

7. A method for detecting a microorganism which comprises:
(a) providing a device which comprises a holder means with an electrode means;
a detection sensor mounted on the holder means in contact with the electrode means, the sensor having spaced apart interdigitated electrodes providing a capacitor and with a capture reagent for the microorganism at least between the electrodes and wherein widths of spaces between each of the electrodes and of the electrodes are each about 5 microns or less, and wherein when the microorganism in a liquid is captured by the capture reagent, the detection sensor is interrogated with an electric circuit producing a change of impedance from the detection sensor without the bound microorganism, thereby detecting the presence of the microorganism;
(b) inserting the detection sensor into a liquid suspected of containing the microorganism;
(c) washing the biosensor to remove any unbound material; and
(d) determining the change of impedance from the detection sensor by interrogating the detection sensor with the electronic circuit to thereby detect the microorganisms.

8. The method of claim 7 wherein the captive reagent is specific for at least a genus classification of the organism and the microorganism is identified at least as to the genus classification.

9. The method of claims 7 or 8 wherein a reference sensor is provided, adjacent to the detection sensor, without the capture reagent for detecting the change of impedance in the presence of the microorganism.

10. A device for detecting an analyte in a solution, which comprises:
a biosensor comprising
(i) a substrate material having a surface;
(ii) an interdigitated array of at least two electrodes disposed on the surface of the substrate wherein a width of a space between the electrodes and of the electrodes are each about 5 microns or less; and
(iii) at least one covalently bound antibody which is specific for the analyte immobilized at least on the surface of the substrate in the space between the electrodes.

11. The device of claim 10 wherein the substrate material is a planar semiconductor device.

12. The device of claim 11 wherein the substrate material is a silicon wafer with an insulator layer passivated thereon.

13. The device of claim 12 wherein the insulator layer is silicon oxide.

14. The device of claim 10 wherein the interdigitated electrodes comprise a metal selected from the group consisting of platinum, palladium, gold, indium tin oxide, iridium, rhodium, osmium, copper, silver, and mixtures thereof.

15. The device of claim 10 wherein a size of the surface between the interdigitated electrodes corresponds to a size range of at least one of the covalently bound antibodies.

16. The device of claim 15 wherein the size is about 4 μm (4000 nm).

17. The device of claim 10 wherein the antibody is immobilized on the surface via a heterobifunctional crosslinker or amine crosslinker.

18. The device of claim 10 wherein the heterobifunctional crosslinker is an N-γ-maleimidobutyryloxy succinimide ester.

19. The device of claim 10 wherein the antibodies are recombinant antibodies.

20. The device of claim 19 wherein the recombinant antibodies are selected from the group consisting of scFV polypeptides and V_{H} chain polypeptides.

21. A method for detecting an analyte in a liquid sample, which comprises:
(a) providing a device comprising a biosensor comprising
(i) a substrate material having a surface;
(ii) an interdigitated array of at least two electrodes disposed on the surface of the substrate wherein widths of a space between the electrodes and of the electrodes are each about 5 microns or less in width; and
(iii) at least one covalently bound antibody which is specific for the analyte immobilized at least on the surface of the substrate in the space between the electrodes; and
(b) immersing the biosensor of the device in the liquid sample for a time sufficient for the antibody to bind the analyte;
(c) removing the biosensor of the device from the liquid sample and immersing the biosensor of the device in a neutral solution; and
(d) applying a current to the biosensor of the device and measuring the impedance of the current wherein an increase in the impedance indicates the liquid sample has the analyte.

22. The method of claim 21 wherein the substrate material is a planar semiconductor device.

23. The method of claim 22 wherein the substrate material is a silicon wafer with an insulator layer passivated thereon.

24. The method of claim 23 wherein the insulator layer is silicon oxide.

25. The method of claim 21 wherein the interdigitated electrodes comprises a metal selected from the group consisting of platinum, palladium, gold, indium tin oxide, iridium, rhodium, osmium, copper, silver, and mixtures thereof.

26. The method of claim 21 wherein a size of the surface between the interdigitated electrodes corresponds to a size range of at least one of the covalently bound antibodies.

27. The method of claim 26 wherein the size is about 4 μm (4000 nm).

28. The method of claim 21 wherein the antibody is immobilized on the surface via a heterobifunctional crosslinker or a homobifunctional crosslinker.

29. The method of claim 21 wherein the heterobifunctional crosslinker is an N-γ-maleimidobutyryloxy succinimide ester.

30. The method of claim 21 wherein the analyte is selected from the group consisting of bacteria, parasites, viruses, toxins, and enzymes.

31. The method of claim 21 wherein the antibodies are recombinant antibodies.

32. The method of claim 31 wherein the recombinant antibodies are selected from the group consisting of scFV polypeptides and V_{H} chain polypeptides.

33. A method for detecting a microorganism in a liquid sample, which comprises:
(a) providing a device comprising a biosensor comprising
(i) a substrate material having a surface;
(ii) an interdigitated array of at least two electrodes disposed on the surface of the substrate wherein widths of a space between the electrodes and of the electrodes are each about 5 microns or less in width;
(iii) at least one covalently bound antibody which is specific for the analyte immobilized at least on the surface of the substrate in the space between the electrodes; and
(b) immersing the biosensor of the device in the liquid sample for a time sufficient for the antibody to bind the microorganism;
(c) removing the biosensor of the device from the liquid sample and immersing the biosensor of the device in a neutral solution; and
(d) applying a current to the biosensor of the device and measuring the impedance of the current wherein an increase in the impedance indicates the liquid sample has the microorganism.

34. A method for identifying a microorganism in a liquid sample, which comprises:
(a) providing a device comprising a biosensor comprising
(i) a substrate material having a surface;
(ii) an interdigitated array of at least two electrodes disposed on the surface of the substrate wherein widths of a space between the electrodes and of the electrodes are each about 5 microns or less; and
(iii) at least one covalently bound antibody which is specific for the analyte immobilized at least on the surface of the substrate in the space between the electrodes; and
(b) immersing the biosensor of the device in the liquid sample for a time sufficient for the antibody to bind the microorganism;
(c) removing the biosensor of the device from the liquid sample and immersing the biosensor of the device in water; and
(d) applying a current to the biosensor of the device and measuring the impedance of the current wherein an increase in the impedance to a particular amount identifies the microorganism.

35. The method of claim 33 or 34 wherein the substrate material is a planar semiconductor device.

36. The method of claim 35 wherein the substrate material is a silicon wafer with an insulator layer passivated thereon.

37. The method of claim 36 wherein the insulator layer is silicon oxide.

38. The method of claim 33 or 34 wherein the interdigitated electrodes comprises a metal selected from the group consisting of platinum, palladium, gold, indium tin oxide, iridium, rhodium, osmium, copper, silver, and mixtures thereof.

39. The method of claim 33 or 34 wherein a size of the surface between the interdigitated electrodes corresponds to a size range of at least one of the covalently bound antibodies.

40. The method of claim 39 wherein the size is about 4 μm (4000 nm).

41. The method of claim 33 or 34 wherein the antibody is immobilized on the surface via a heterobifunctional crosslinker or a homobifunctional crosslinker.

42. The method of claim 33 or 34 wherein the heterobifunctional crosslinker is an N-γ-maleimidobutyryloxy succinimide ester.

43. The method of claim 33 or 34 wherein the support means is an elongated probe.

44. The method of claim 33 or 34 wherein the microorganism is a bacterium.

45. The method of claim 44 wherein the bacterium is Escherichia coli strain O157:H7.

46. The method of claim 33 or 34 wherein the antibodies are recombinant antibodies.

47. The method of claim 46 wherein the recombinant antibodies are selected from the group consisting of scFV polypeptides and V_{H} chain polypeptides.
